(19) United States
(12) Patent Application Publication
(10) Pub. No.: US 2012/0016267 A1
(43) Pub. Date: Jan. 19, 2012

(54) TECHNIQUES FOR PROVIDING HAND-BASED PERSONALITY INVENTORIES

(76) Inventor: Edward Dickson Campbell, Seattle, WA (US)

(21) Appl. No.: 12/837,277

(22) Filed: Jul. 15, 2010

(51) Int. Cl. A61B 5/103

(52) U.S. Cl. 600/587

(57) ABSTRACT

Techniques described and suggest herein include systems and methods for providing to a user an inventory set of personality characteristics derived from hand characteristics. In an embodiment, providing the hand characteristics includes finding mismatched personality types derived from the hand characteristics, extrapolating new personality characteristics derived from the hand characteristics, and updating the inventory set of personality characteristics. In a further embodiment, the extrapolating is done using a context of the hand characteristics.

### BACKGROUND

An individual's personality has always been vitally important to success. Whether it be personal success or the success of the companies for which an individual works, matching a personality type to conducive circumstances tends to yield positive results. Further, understanding the competitive and deficient aspects of a personality can allow for appropriate allocation of time and resources to leveraging strengths and developing weaknesses. Indicators that help to identify personality types in others can also be used for widespread practical applications. These applications may range from selecting an appropriate employee for particular company tasks to matching personality types that show the best possibility for a lasting relationship.

### BRIEF SUMMARY

The following presents a simplified summary of some embodiments of the invention in order to provide a basic understanding of the invention. This summary is not an extensive overview of the invention. It is not intended to identify key/critical elements of the invention or to delineate the scope of the invention. Its sole purpose is to present some embodiments of the invention in a simplified form as a prelude to the more detailed description that is presented later.

In an embodiment, a computer-implemented method for providing to a user an inventory set of personality characteristics derived from hand characteristics is disclosed. Hand characteristics, in an embodiment, include morphological characteristics and/or measurement characteristics. A morphological characteristic, in an embodiment, is a characteristic of a hand's form and/or shape. A measurement characteristic, in an embodiment, is a characteristic of a hand that is based at least in part of a physical measurement of a hand. Measurements include, but are not limited to dimensions of the hands or parts thereof, measurements of ranges of motion, measurements of responses to physical stimulus, and the like. In an embodiment, a plurality of personality characteristics are stored in a data store in a manner that associates the personality characteristics with corresponding hand characteristics. Information, based at least in part on a subject's hand, that identifies a set of hand characteristics is received. A set of personality characteristics, based at least in part on the received information, are identified from the data store. Information that is based at least in part on the identified set of personality characteristics is provided for display by the user.

In an embodiment, the hand characteristics further comprise one or more of the following: left or right hand; hand dominance; dermatoglyphic patterns for one or more fingers; finger flexibility for one or more fingers; finger placement at rest for one or more fingers; finger morphology for one or more fingers; finger absolute dimensions for one or more fingers; finger relative dimensions for one or more fingers; and finger nail dimensions for one or more fingers.

In an embodiment, the set of personality characteristics identified is further described. Mismatched personality characteristics may be identified based at least in part on hand characteristics. New personality characteristics based on least in part on the hand characteristics may be extrapolated and a set of personality characteristics may be updated. In addition, new personality characteristics extrapolated are based at least in part on a context of the hand characteristics.

Hand characteristics may be received from a user interface comprising one or more forms and/or from at least one machine designed to determine hand characteristics. A three-dimensional modeling algorithm may be used to model hand characteristics.

In an embodiment, the set of personality characteristics further comprise an inventory set of personality characteristics. Dermatoglyphic patterns may be received from visual recording, mechanical recording, optical recording, electrical recording, ultrasound recording, thermal recording, or other devices In one embodiment, finger flexibility characteristics are received from a pressure glove.

In another embodiment, a computer-readable storage medium includes instructions that, when executed by one or more processors, cause the one or more processors to store, in a data store, a plurality of personality characteristics in a manner that associates the personality characteristics with corresponding hand characteristics; receive, based at least in part on a subject's hand, information that identifies a set of hand characteristics; identify, based at least in part on the received information, a set of personality characteristics from the data store; and provide for display to the user information that is based at least in part on the identified set of personality characteristics.

As noted, the hand characteristics may comprise one or more of: identification of left or right; hand dominance; dermatoglyphic patterns for one or more fingers; finger flexibility for one or more fingers; finger placement at rest for one or more fingers; finger morphology for one or more fingers; finger absolute dimensions for one or more fingers; finger relative dimensions for one or more fingers; and nail dimensions for one or more fingers. The hand characteristics may include one or more measurements based on a physical test of a hand. The instructions may further include instructions that cause the one or more processors to identify mismatched personality characteristics identified based at least in part on the hand characteristics; extrapolate new personality characteristics based at least in part on the hand characteristics; and update the plurality of personality characteristics in the data store. In addition, the instructions may further include instructions that cause the one or more processors to provide a series of interface elements for receiving the hand characteristics, where at least one of the interface elements is conditional on input received by one or more of the interface elements.

In an embodiment, a computer system for providing a personality inventory is disclosed. The computer system, in an embodiment, includes a data store that stores a plurality of personality characteristics in a manner that associates the personality characteristics with corresponding hand characteristics; one or more processors; and memory, including instructions that, when executed by the one or more processors, cause the one or more processors to: receive, based at least in part on a subject's hand, information that identifies a set of hand characteristics; identify, based at least in part on the received information, a set of personality characteristics from the data store; and provide for display to the user information that is based at least in part on the identified set of personality characteristics.

The hand characteristics that the computer system receives may include one or more of: identification of left or right; hand dominance; dermatoglyphic patterns for one or more fingers; finger flexibility for one or more fingers; finger placement at rest for one or more fingers; finger morphology for one or more fingers; finger absolute dimensions for one or more fingers; finger relative dimensions for one or more fingers; and nail dimensions for one or more fingers. In one embodiment, the hand characteristics comprise a plurality of the aforementioned characteristics. The instructions may further include instructions that cause the one or more processors to provide a plurality of interface elements for receiving the hand characteristics, at least one of the interface elements being conditional on input received by one or more of the interface elements. The data store may associate one or more of the hand characteristics with honesty.

For a fuller understanding of the nature and advantages of the present invention, reference should be made to the ensuing detailed description and accompanying drawings.

### BRIEF DESCRIPTION OF THE DRAWINGS

FIG. 1 shows a right hand with an example of a labeling of features thereof, in accordance with an embodiment.

FIG. 2 shows a left hand with an example of a labeling of features thereof, in accordance with an embodiment.

FIG. 3 shows a left hand with an example labeling of the relative anatomical directions for the hand in accordance with an embodiment.

FIG. 4 shows a dermatoglyphic loop pattern in accordance with an embodiment.

FIG. 5 shows a dermatoglyphic double loop pattern in accordance with an embodiment.

FIG. 6 shows a dermatoglyphic composite loop pattern in accordance with an embodiment.

FIGS. 7a-7b and 8a-8b show a 12:00 extending dermatoglyphic loop pattern in accordance with an embodiment.

FIG. 9a-9b shows a 1:00 extending dermatoglyphic loop pattern in accordance with an embodiment.

FIG. 10a-10b shows a 11:00 extending dermatoglyphic loop pattern in accordance with an embodiment.

FIGS. 11a-11b shows a 2:00 extending dermatoglyphic loop pattern in accordance with an embodiment.

FIG. 12a-12b shows a 10:00 extending dermatoglyphic loop pattern in accordance with an embodiment.

FIG. 13a-13b shows a 9:00 extending dermatoglyphic loop pattern in accordance with an embodiment.

FIG. 14a-14b shows a 3:00 extending dermatoglyphic loop pattern in accordance with an embodiment.

FIG. 15 shows a dermatoglyphic target whorl pattern in accordance with an embodiment.

FIG. 16 shows a dermatoglyphic spiral whorl pattern in accordance with an embodiment.

FIG. 17 shows a dermatoglyphic composite whorl pattern in accordance with an embodiment.

FIG. 18 shows a dermatoglyphic elongated whorl pattern in accordance with an embodiment.

FIG. 19 shows a dermatoglyphic composite whorl pattern in accordance with an embodiment.

FIG. 20 shows a dermatoglyphic simple arch pattern in accordance with an embodiment.

FIG. 21 shows a dermatoglyphic tented arch pattern in accordance with an embodiment.

FIG. 22 shows a dermatoglyphic pocket loop pattern in accordance with an embodiment.

FIG. 23 shows a dermatoglyphic arch loop pattern in accordance with an embodiment.

FIG. 24 shows a dermatoglyphic arch whorl pattern in accordance with an embodiment.

FIGS. 25-27 show accidental dermatoglyphic patterns in accordance with an embodiment.

FIGS. 28 and 29 show broken dermatoglyphic patterns in accordance with an embodiment.

FIG. 30 shows an example of high finger flexibility, between 45° and 90° in accordance with an embodiment.

FIG. 31 shows a fully extended thumb in accordance with an embodiment.

FIG. 32 shows a bent thumb in accordance with an embodiment.

FIG. 33 shows a right hand with an index finger thumb angle of over 90° in accordance with an embodiment.

FIG. 34 shows a left hand with an index finger thumb angle of over 90° in accordance with an embodiment.

FIG. 35 shows a left hand with an index finger thumb angle of under 90° in accordance with an embodiment.

FIG. 36 shows closed fingers, with the thumb tucked into the closed fingers, in accordance with an embodiment.

FIGS. 37 and 38 show curled fingers in accordance with an embodiment.

FIG. 39 shows fully extended and relaxed fingers in accordance with an embodiment.

FIG. 40 shows a separated little finger in accordance with an embodiment.

FIG. 41 shows a separated index finger in accordance with an embodiment.

FIGS. 42-44 show a prominent proximal thumb knuckle in accordance with an embodiment.

FIG. 45 shows an example of various prominent proximal knuckles in accordance with an embodiment.

FIG. 46 shows a prominent proximal index finger knuckle in accordance with an embodiment.

FIG. 47 shows a prominent proximal middle finger knuckle in accordance with an embodiment.

FIG. 48 shows a prominent proximal ring finger knuckle in accordance with an embodiment.

FIG. 49 shows a prominent proximal little finger knuckle in accordance with an embodiment.

FIGS. 50 and 51 show an example of various prominent medial knuckles in accordance with an embodiment.

FIG. 52 shows a prominent medial index finger knuckle in accordance with an embodiment.

FIG. 53 shows a prominent medial middle finger knuckle in accordance with an embodiment.

FIG. 54 shows a prominent medial ring finger knuckle in accordance with an embodiment.

FIG. 55 shows a prominent medial little finger knuckle in accordance with an embodiment.

FIG. 56 shows an example of a prominent distal knuckle in accordance with an embodiment.

FIG. 57 shows a prominent distal index finger knuckle in accordance with an embodiment.

FIG. 58 shows a prominent distal middle finger knuckle in accordance with an embodiment.

FIG. 59 shows a prominent distal ring finger knuckle in accordance with an embodiment.

FIG. 60 shows a prominent distal little finger knuckle in accordance with an embodiment.

FIG. 61 shows an example of swollen nail phalanges in accordance with an embodiment.

FIGS. 62 and 63 show an index finger shorter than the nail base of a middle finger, in accordance with an embodiment.

FIGS. 64 and 65 show an index finger longer than a ring finger, in accordance with an embodiment.

FIGS. 66 and 67 show an index finger shorter than a ring finger, in accordance with an embodiment.

FIG. 68 shows an example of an average size thumb in accordance with an embodiment.

FIGS. 69-71 show a dominant thumb in accordance with an embodiment.

FIGS. 72 and 73 show balanced index and ring fingers in accordance with an embodiment.

FIGS. 74-76 show a medially bent index finger in accordance with an embodiment.

FIG. 77 shows a laterally bent index finger in accordance with an embodiment.

FIGS. 78 and 79A show a medially bent middle finger in accordance with an embodiment.

FIG. 79B shows a laterally bent middle finger in accordance with an embodiment.

FIGS. 80 and 81 show an extra long middle finger in accordance with an embodiment.

FIG. 82 shows a laterally bent ring finger in accordance with an embodiment.

FIGS. 83 and 84 show a medially bent middle finger and a laterally bent ring finger in accordance with an embodiment.

FIG. 85 shows a medially bent ring finger in accordance with an embodiment.

FIGS. 86 and 87 show a long little finger in accordance with an embodiment.

FIGS. 88-90 show an average length little finger in accordance with an embodiment.

FIGS. 91 and 92 show a small little finger medial phalange in accordance with an embodiment.

FIGS. 93 and 94 show a crooked little finger in accordance with an embodiment.

FIG. 95 shows an example of various nail lengths in accordance with an embodiment.

FIGS. 96-98 show a short nail in accordance with an embodiment.

FIG. 99 shows an average length nail in accordance with an embodiment.

FIG. 100 shows a long length nail in accordance with an embodiment.

FIG. 101 is a simplified block diagram of a computer system that may be used to practice an embodiment of the present invention.

FIGS. 102A-C show a process for providing a set of personality characteristics derived from hand characteristics in accordance with an embodiment.

FIG. 103 shows a general example “Hand Characteristics” User Interface page in accordance with an embodiment.

FIG. 104 shows a Hand Type User Interface page in accordance with an embodiment.

FIG. 105 shows a Dermatoglyphic Patterns User Interface page in accordance with an embodiment.

FIG. 106 shows a Finger Flexibility User Interface page in accordance with an embodiment.

FIG. 107 shows a Finger Morphology User Interface page in accordance with an embodiment.

FIG. 108 shows a Knuckle Dimensions User Interface page in accordance with an embodiment.

FIG. 109 shows a Relative Finger Length User Interface page in accordance with an embodiment.

FIG. 110 shows a Finger Dimensions: Size User Interface page in accordance with an embodiment.

FIG. 111 shows a Finger Dimensions: Shape User Interface page in accordance with an embodiment.

FIG. 112 shows a Nail Size User Interface page in accordance with an embodiment.

FIG. 113 shows the Finger, Pattern, Arch, Loop, Whorl, and Composite database tables in accordance with an embodiment.

FIG. 114 shows the database tables that associate personality characteristics with dermatoglyphic patterns of the thumb in accordance with an embodiment.

FIG. 115 shows the database tables that associate personality characteristics with dermatoglyphic patterns of the index finger in accordance with an embodiment.

FIG. 116 shows the database tables that associate personality characteristics with dermatoglyphic patterns of the middle finger in accordance with an embodiment.

FIG. 117 shows the database tables that associate personality characteristics with dermatoglyphic patterns of the ring finger in accordance with an embodiment.

FIG. 118 shows the database tables that associate personality characteristics with dermatoglyphic patterns of the little finger in accordance with an embodiment.

FIG. 119 shows the interaction process between an application server and a data store in accordance with an embodiment.

FIGS. 120-123 show short-hand code for hand characteristics in accordance with an embodiment.

FIG. 124 shows a simplified block diagram of a computer system that may be used to practice an embodiment of the present invention.

### DETAILED DESCRIPTION

In the following description, various embodiments of the present invention will be described. For purposes of explanation, specific configurations and details are set forth in order to provide a thorough understanding of the embodiments. However, it will also be apparent to one skilled in the art that the present invention may be practiced without the specific details. Furthermore, well-known features may be omitted or simplified in order not to obscure the embodiment being described.

Various techniques described and suggested herein include systems and methods for providing a set of personality characteristics derived from a set of hand characteristics. Techniques described herein may be used, for instance, to identify personality characteristics by examining a subject's hand to identify hand characteristics, and using those hand characteristics to determine a personality profile from information stored in a data store. Hand characteristics that may utilized are further described in the following paragraphs.

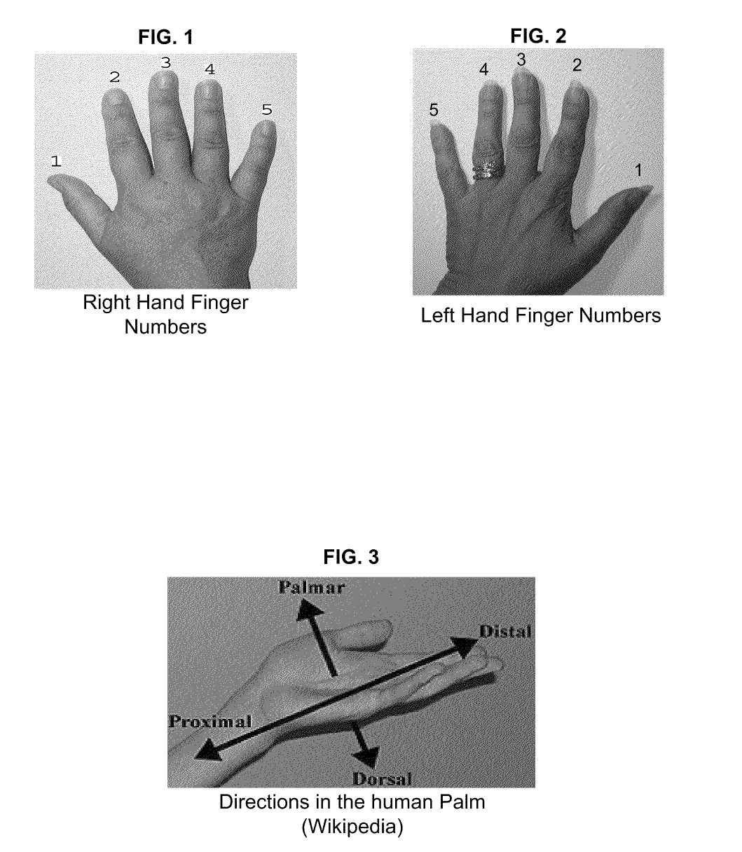

FIG. 1 shows an illustrative example of a labeling of the right hand of a sample subject. In this example, the fingers of the right hand are labeled as follows: Thumb 1; Index 2; Middle 3; Ring 4; and Little 5. As shown in FIG. 2, which shows an illustration of a subject's left hand, a similar numbering can be applied to the left hand.

FIG. 3 shows a left hand oriented such that the palm is facing upward. In FIG. 3, anatomical directions are labeled that will be used in the descriptions hereafter. In particular, a Palmar direction is shown as being a direction extending approximately perpendicularly from a plane formed by the palm of the hand, the direction extending away from the palm side of the hand. A Dorsal direction is shown as being parallel to, but opposite the Palmar direction. A Proximal direction is shown as being a direction extending within the plane formed by the palm of the hand towards the wrist. A Distal direction is shown as being parallel, but opposite to the Proximal direction.

In an embodiment, hand characteristics are used to derive personality characteristics for a subject. Hand characteristics may include, but are not limited to, dermatoglyphic patterns, finger flexibility, finger morphology, knuckle dimensions, relative finger length, finger dimensions, and any others suitable observations. Example hand characteristic and example associated personality characteristics are further described below.

In an embodiment, the context of a hand characteristic may be utilized to derive additional personality characteristics. For example, a context for an epidermal pattern may be a finger, a particular part of a finger, a particular part of the palm, or any other information that shows the physiological properties of the epidermal pattern. In an embodiment, a hand characteristic and a context are associated with personality characteristics for a subject.

Dermatoglyphic Patterns

Dermatoglyphic patterns are patterns created by the valleys and ridges of the epidermis of the hand. Dermatoglyphic patterns may include patterns on the fingertips, the palm, and other locations of the hand. These patterns, in an embodiment, are classified according to one or more pattern types, such as loops or whorls. The context of a pattern can be a major differentiating factor. For instance, in an embodiment, patterns are associated with different personality characteristics for the right and left hands.

Patterns on the right hand, in an embodiment, are associated with a subject's behavior and honesty in dealing with those who are not perceived as family members, loved ones or extended family members. Examples include behavior when shopping, driving, working, or interactions with strangers, rivals and enemies. Patterns on the left hand, in an embodiment, are associated with the subject's behavior and honesty in dealing with those who are not perceived as strangers, potential rivals or enemies but as loved ones, close family or extended family members. Examples of such people include a spouse, parents, grand parents, siblings, uncles, aunts, close cousins and those residing in the same home or group.

Patterns may also be associated with different categories of personality characteristics for each finger. For instance, in an embodiment, the thumb may correspond to action and thought processes related to immediate attention and action. The index finger may correspond to ego, and thought processes related to concepts, goals, planning and organization. The middle finger may correspond to balance, and thought processes related to judgment together with related moods. The ring finger may correspond to work effort, including single and multiprocess focusing. And the little finger may correspond to communication, and thought processes related to patterns, math, business and finance, and (when on the left hand) intimate communications.

There are a number of different types of dermatoglyphic patterns. The following paragraphs present explanations for a sample set of patterns. These explanations are only examples and other patterns may be utilized.

Loop Pattern

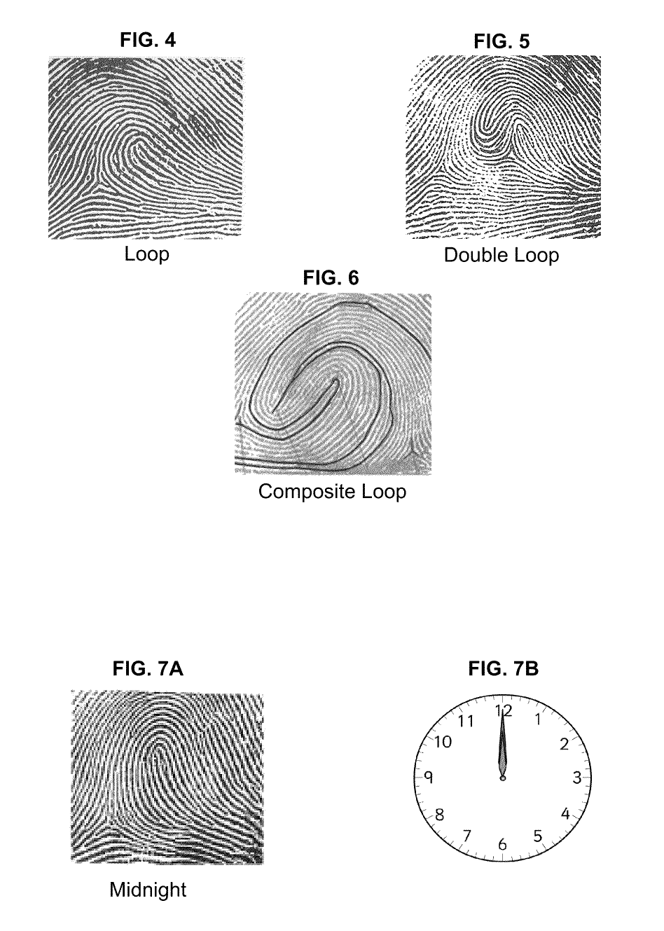

FIG. 4 shows an example of a loop pattern. Loop patterns, in an embodiment, are of three types, those with the closed end facing the thumb (Ulnar), those with the closed end facing the little finger (Radial), and those that comprise a series of two side by side loops, one usually looping around the other. For the third type of loop, the base of the two loops may determine whether they are radial or ulnar. If the base is on the little finger side, in an embodiment, the loops are categorized as ulnar. If the base is on the thumb side, in an embodiment, they are categorized as radial. FIG. 5 shows an example of a third type of loop labeled as a double loop, where two loops form adjacent one to another. FIG. 6 shows an example of a third type of loop labeled as a composite loop, where one loop curves so as to form another loop.

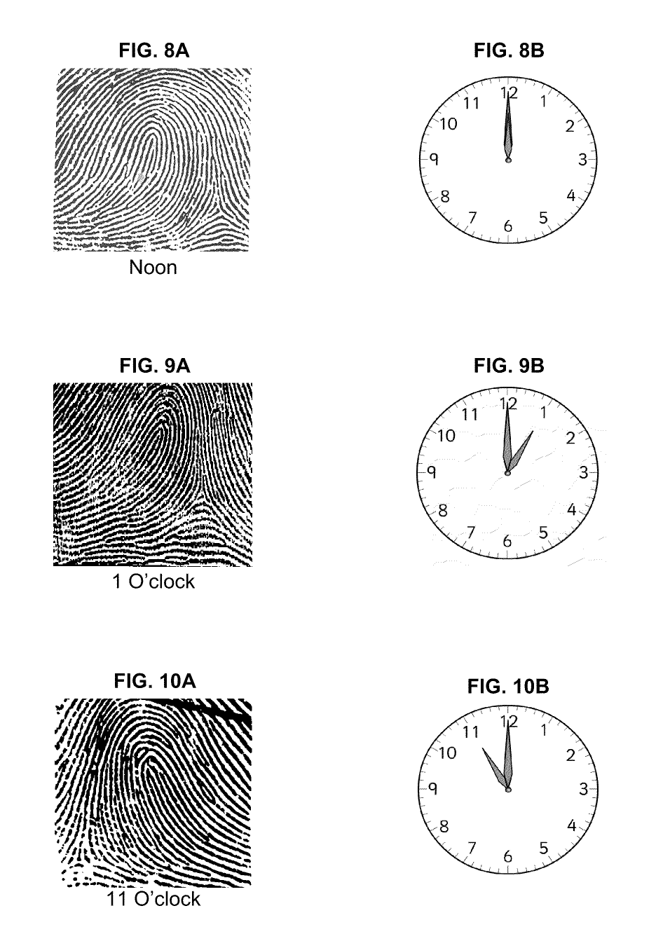

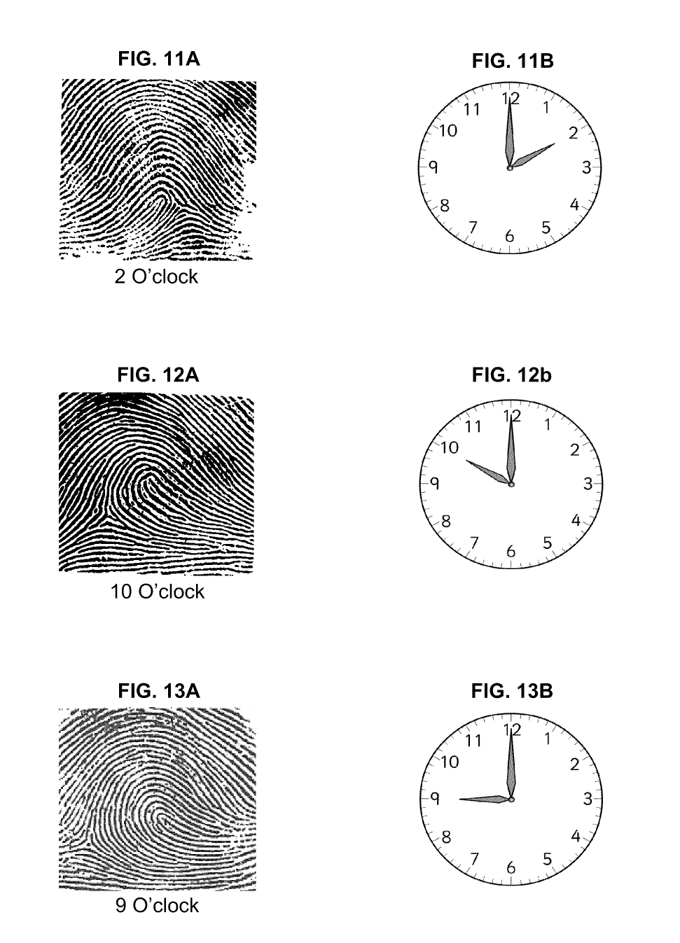

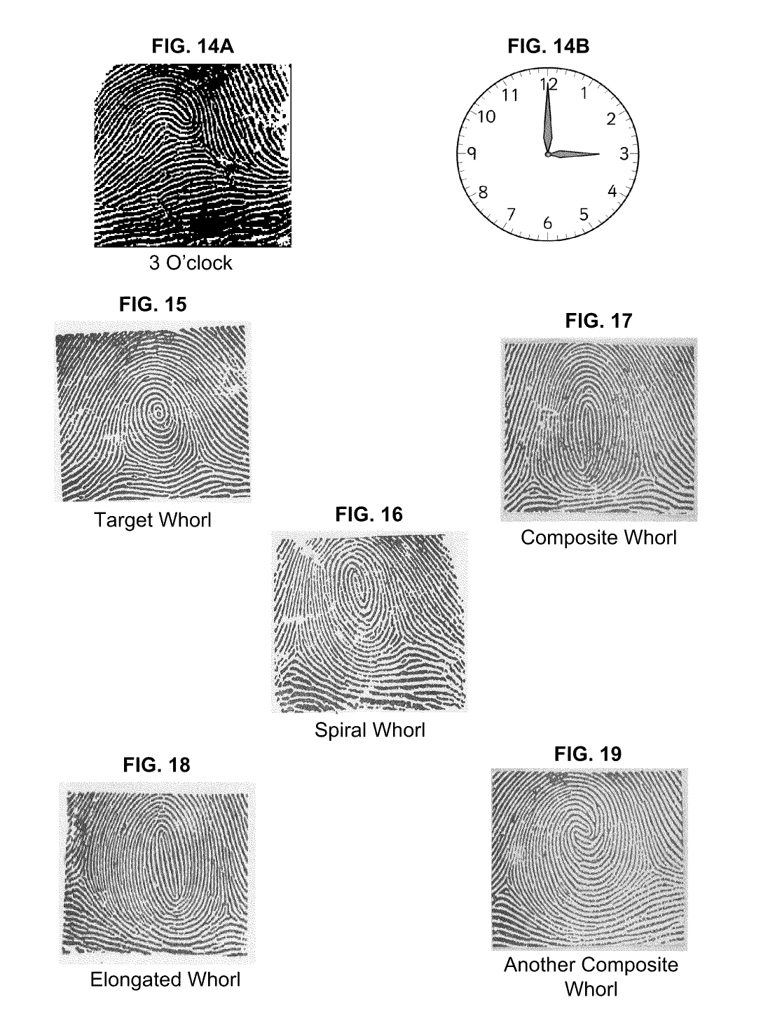

In an embodiment, the direction a loop extends, labeled as the direction an hour hand extends for a particular hour on an analog clock (i.e. 12:00), as determined by the final extent of the loop that is parallel to the tip of the finger and distal phalange crease, may indicate a corresponding degree of honesty. A loop extending towards 12:00, as shown in FIGS. 7 and 8, in an embodiment, may be associated with brutal honesty. A loop extending towards 11:00 or 1:00, as shown in FIGS. 9 and 10, in an embodiment, may be associated with kind honesty. A loop extending towards 10:00 or 2:00, as shown in FIGS. 11 and 12, in an embodiment, may be associated with commercial honesty. And a loop extending towards 9:00 or 3:00, as shown in FIGS. 13 and 14, in an embodiment may, be associated with convenient honesty. A brutally honest person has great difficulty even trying to lie. A brutally honest person's way to protect others is to stay silent. If asked, a brutally honest person will reply as he or she sees or understands it. Also, a brutally honest person may not be accurate or complete, but will be truthful. A kindly honest person will generally be truthful except where truth may hurt someone else that they know, such as by hurting their feelings. Kindly honest people may tell “white” lies. A commercially honest person will be as honest as expected in the business or diplomatic affairs. If a risk taker, a commercially honest person will run a bluff in a poker game. Commercially honest people also generally protect trade secrets. Caveat Emptor.

Loop patterns that appear on the 2nd, index finger, in an embodiment, reflect what they seek in life. For example, the brutally honest pattern will reflect a life seeking honesty and truth.

When a loop pattern appears on a person's 1st finger or thumb, the person may act with others in the manner indicated, but without competition unless the pattern is part of a pocket loop in which case they may act either in cooperation or competition as the circumstances dictate. A conveniently honest person is honest when it suits him or her.

In an embodiment, loops may also be associated with scanner, lookout, or multiprocessor type personality characteristics. For instance, people having loops, in an embodiment, are identified as people who pay Attention to the immediate environment, who are application oriented, who are immediate responders, who plans dinner at dinner time, and the like. Ulnar loops, in an embodiment, may indicate an attention to the world and radial loops, in an embodiment, may indicate an attention to the self. Double loops, in an embodiment, may maintain the indications of ulnar and radial loops, and may also indicate multi-parameter analysis when assessing a situation. Composite loops, in an embodiment, may add a competitive drive to the indications of a double loop.

Whorl Pattern

FIGS. 15-19 show examples of whorl patterns. In an embodiment, a whorl may be a circular pattern, sometimes comprising or approximating concentric circles or elongated circles. FIG. 15 shows an example of a target whorl, comprising concentric circles formed by the ridges. FIG. 16 shows an example of a spiral whorl, where ridges of the pattern form a spiral. FIG. 18 shows an example of an elongated whorl, where the ridges approximate concentric ellipses. And FIGS. 17 and 19 show examples of composite whorls, which also may be labeled as incomplete or yin/yang whorls.

In an embodiment, whorls may indicate goal setting and planning. For instance, a person with a whorl pattern may be identified as a “future tripper.” A target whorl, in an embodiment, may indicate specific focus from one perspective while a spiral whorl, in an embodiment, may indicate a specific focus from more than one perspective. An elongated whorl, in an embodiment, may indicate a focus on the big picture with enhanced deliberation. A composite or incomplete whorl, in an embodiment, may indicate a need to double check and an ability to discover answers not readily apparent from linear thinking as well as an ability for rapid reassessment and rapprochement.

Arch Pattern

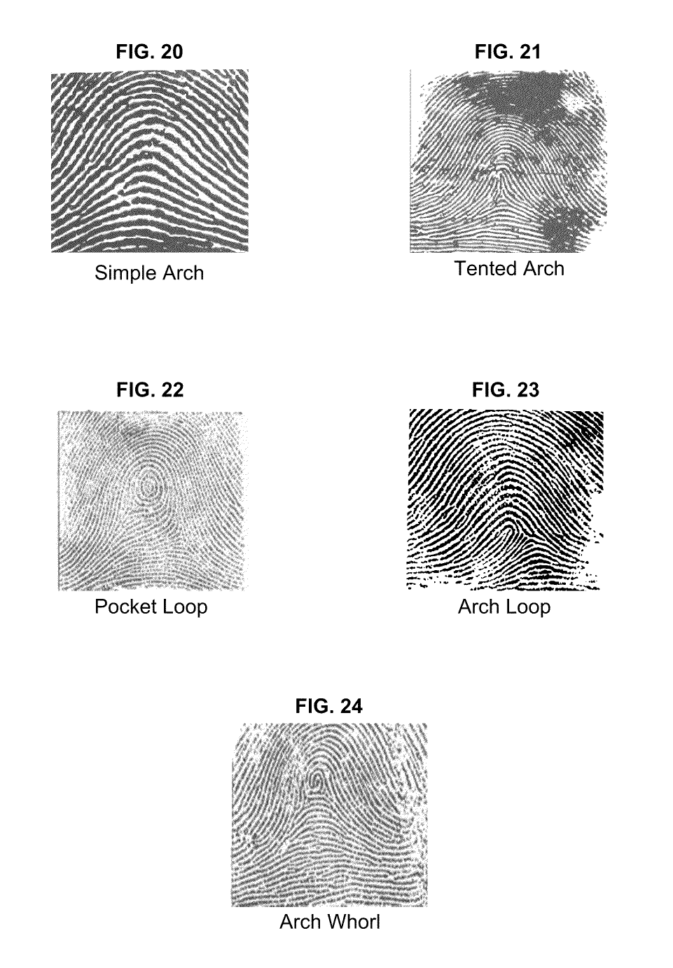

FIGS. 20 and 21 show examples of an arch pattern. An arch pattern, in an embodiment, is a pattern where the ridges resemble a bell curve, or a wave pattern. FIG. 20 shows an example of a simple arch, where the ridges approximate bell curves, and FIG. 21 shows an example of a tented arch, where the ridges approximate triangles.

In an embodiment, arches indicate characteristics often associated with engineer. Thus, in an embodiment, people having arches are identified as people who engage in planning followed by sustained effort or project oriented personality characteristics. A tented arch, in an embodiment, is associated with cheerleading type personality characteristics that may indicate sustained enthusiastic effort and open honesty.

Compound Pattern

FIG. 22 shows an example of a compound pattern. A compound or composite pattern, in an embodiment, may be a combinations of any two patterns. An example of a combination is the loop and whorl, labeled as a pocket loop, pocket whorl or a peacock's eye, as shown in FIG. 22. Other examples of compound patterns include the loop arch, where a few ridges forming a loop pattern are below or embodied within ridges forming an arch pattern, as exemplified in FIG. 23. An arch whorl, where ridges may form a whorl inside of an arch pattern, is exemplified in FIG. 24.

In an embodiment, compound patterns may indicate a combination of personality characteristics determined by the patterns that compose them. For instance, a peacock's eye, in an embodiment, may combine one or more aspects of the personality characteristics indicated by a loop with one or more aspects of the personality characteristics indicated by a whorl.

Complex or Accidental Patterns

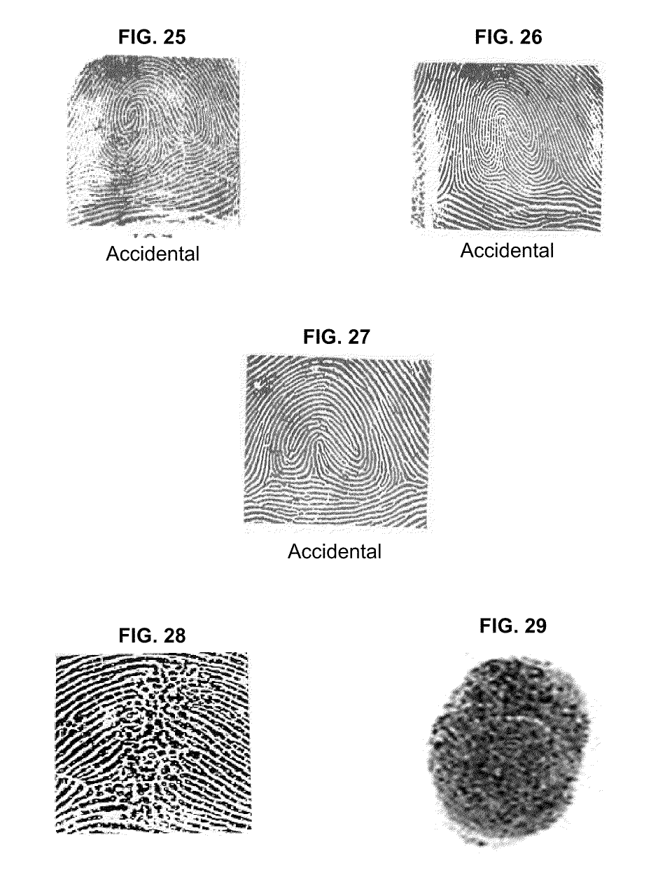

FIGS. 25-27 show examples of complex or accidental patterns. Complex or accidental patterns are rare and may appear in a number of ways. Complex or accidental prints, in an embodiment, do not have associated personality characteristics, although they may.

Broken Pattern

In an embodiment, FIG. 28 shows an example of a broken pattern. A broken pattern, in an embodiment, is a pattern where some adjacent ridges are incompletely formed preventing an otherwise classified pattern from fully forming. A broken pattern may indicate intermittent attention disorders such as ADD and ADHD.

No Pattern

FIG. 29 shows an example of a no print pattern. The medical names for true no prints are Naegeli-Franceschetti-Jadassohn syndrome (NFJS) and dermatopathia pigmentosa reticularis (DPR). These two closely relate to the autosomal dominant ectodermal dysplasia syndromes that clinically share a complete absence of dermatoglyphic patterns, a reticulate pattern of skin hyperpigmentation, a thickening of the palms and soles (palmoplantar keratoderma), abnormal sweating, and other subtle developmental anomalies of the teeth, hair, and skin. They are also known as “Dermatopathia pigmentosa reticularis hyperkeratotica et mutilans,” “Dermatopathia pigmentosa reticularis hypohidotica et atrophica,” and “Dermatopathic pigmentosareticularis.” No print is very rare and, in an embodiment, does not have associated personality characteristics, although it may.

In an embodiment, one or more of the dermatoglyphic patterns, context, and any associated personality characteristics, and other similar variants, are utilized to identify a set of personality characteristics for a subject.

While the foregoing provides examples of behavioral correspondences, in an embodiment, fingerprints have no behavioral correspondences without relationship to the finger and hand where found. For instance, while loops can be related to scanning, whorls can be related to focus (macroscopic or microscopic) and arches can be related to effort, and combination prints can be related to a combination of these traits, and while other prints may be related to some forms of genetic or congenital anomaly, until they are related to specific fingers and specific hands, no particular behavioral correspondences can be safely identified. Additionally, combined prints, in an embodiment, combine characteristics. Thus, in an embodiment, a pocket whorl is associated with a person that is a potential cooperative competitor. A more thorough set of behavioral correspondences, in accordance with an embodiment, is provided below.

Finger Flexibility

Finger flexibility refers to the range of motion for the fingers of the hand. The ranges of motion measured, in an embodiment, may include the flexibility of the fingers when pressed back, the flexibility of the thumb, the flexibility of the fingers relative to each other, or other similar variants.

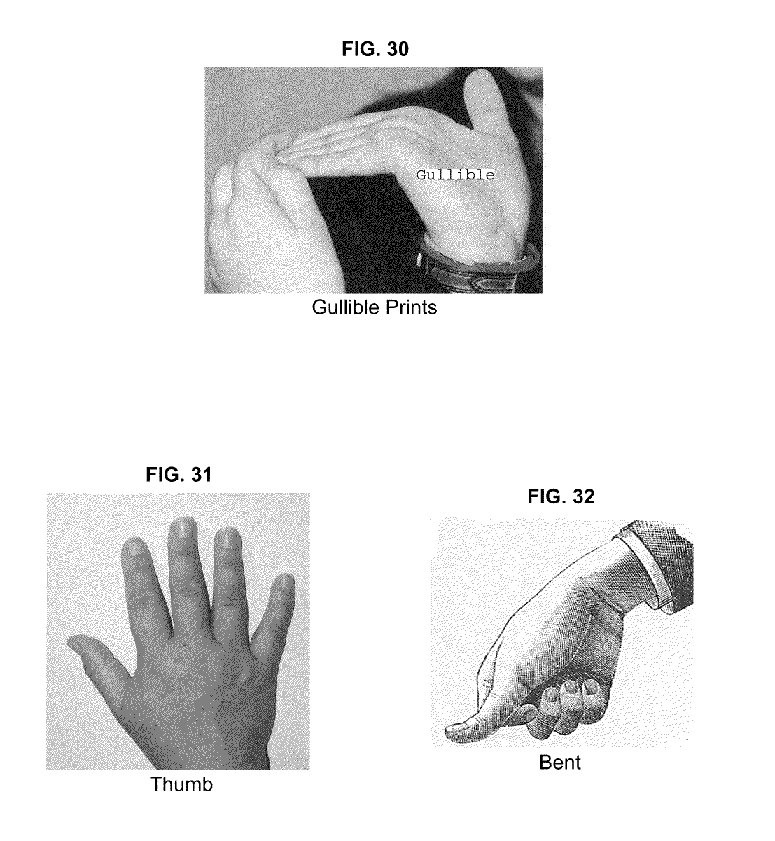

In an embodiment, the flexibility of the index, middle, ring, and little fingers may indicate an open mind. The flexibility is tested, in an embodiment, by applying slight pressure pushing extended fingers back, against their maximum rotational position, towards the wrist, as shown in FIG. 30. High flexibility, in an embodiment, which is an angle from 45° to 90° with respect to the vertical axis of the palm, as shown in FIG. 30, may indicate a gullible person. Moderate flexibility, in an embodiment, which is an angle from 20° to 45°, may indicate an open mind. Low flexibility, in an embodiment, which is an angle from less than 20°, may indicate a closed mind, one with very fixed opinions.

In an embodiment, the stiffness of the thumb may indicate a degree of stubbornness. Flexibility may be tested, in an embodiment, on a fully extended thumb, as shown in FIG. 31, by applying slight pressure pushing the thumb backwards. Little or no flexibility of the thumb, in an embodiment, may indicate stubbornness. Moderate flexibility of the thumb, in an embodiment, as shown in FIG. 32, may indicate a flexible mind, open to persuasion.

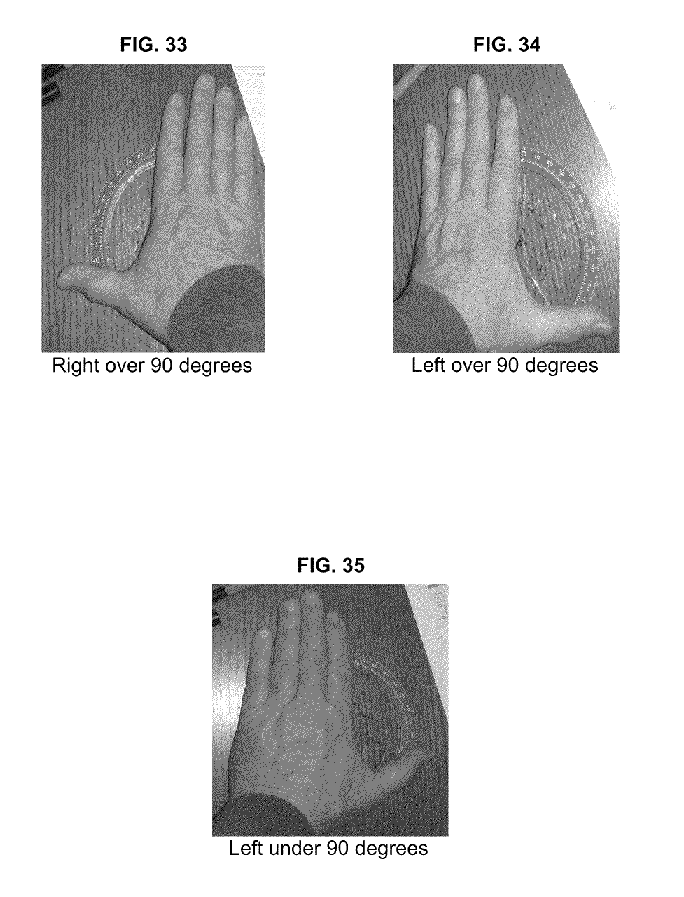

In an embodiment, the angle between the thumb and index finger may indicate a degree of co-dependency. Flexibility may be tested, in an embodiment, by laying the subject's hand flat on a surface and gently pulling the thumb away from the index finger until resistance is met. An angle of less than 90° for both hands, in an embodiment, as shown in FIG. 35, may indicate no co-dependency has ever existed. An angle of 90°, as shown in FIG. 33, or over 90°, as shown in FIG. 34, for both hands, in an embodiment, may indicate an established co-dependency. An angle of less than 90° for one hand but over 90° for the other hand, in an embodiment, may indicate a co-dependency has been overcome.

In an embodiment, one or more ranges of motion, context, and any associated personality characteristics, and other similar variants, may be utilized to identify a set of personality characteristics for a subject.

Finger Morphology

Finger morphology is an observation of the structure and form of the fingers. Finger morphologies may include, in an embodiment, the manner in which a subject holds their fingers, an observed separation between the fingers in a relaxed state, and other similar variants. In an embodiment, measurements regarding the flexibility of fingers, their placement at rest, their shapes and lengths, including relative finger lengths and comparative lengths and sizes of phalanges, and prominence of knuckles, if any, are taken and associated with various behavioral characteristics, as described herein.

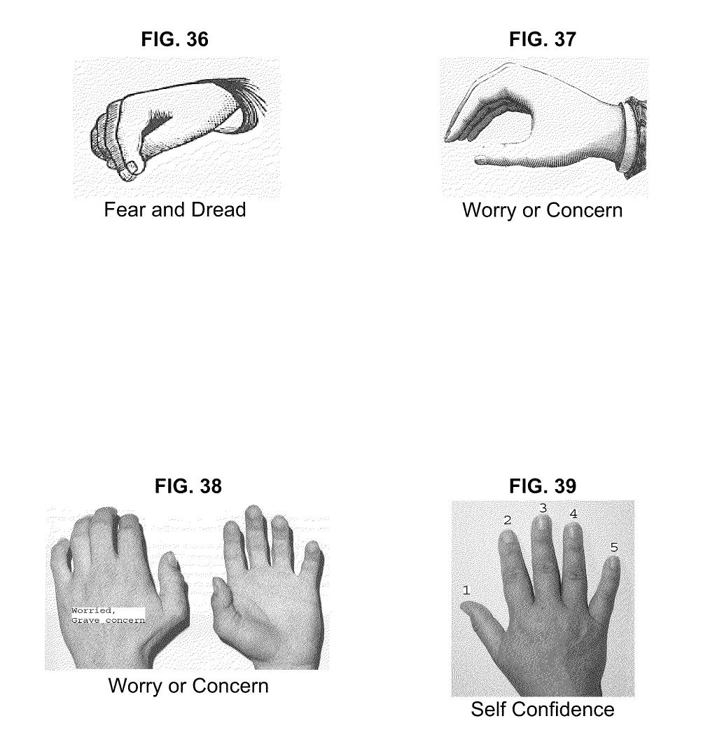

In one embodiment, finger morphology is tested by asking the subject to lay their palms down on a flat surface. Closed fingers, with the thumb tucked into the closed fingers, as shown in FIG. 36, in an embodiment, may indicate a mental impression of fear or stress. Curled fingers, without the thumb tucked, as shown in FIGS. 37 and 38, in an embodiment, may indicate a mental impression of worry or concern. Fully extended fingers in a relaxed state, as shown in FIG. 39, in an embodiment, may indicate a mental impression of self confidence or lack of concern.

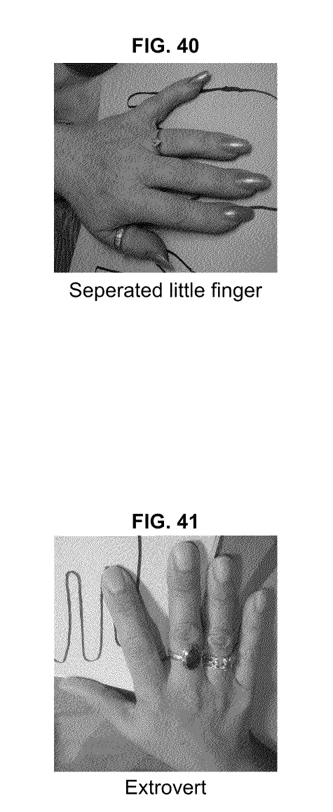

In one embodiment, the natural positioning of the fingers when fully extended on a flat surface may also indicate additional personality characteristics. A little finger separated from the rest of the fingers, in an embodiment, as shown in FIG. 40, may indicate a need for space. Little finger separation on the right hand, in an embodiment, may indicate a need for space at work or in school. Little finger separation on the left hand, in an embodiment, may indicate a need for space at home or in a subject's personal life. An index finger separated from a middle finger, in an embodiment, as shown in FIG. 41, may indicate a need for attention or recognition. Index finger separation on the right hand, in an embodiment, may indicate extroverted characteristics, such as a need for recognition at work or in school. Index finger separation on the left hand, in an embodiment, may indicate a need for recognition at home or in a subject's personal life.

In an embodiment, one or more finger morphologies, context, and any associated personality characteristics, and other similar variants, may be utilized to identify a set of personality characteristics for a subject.

Knuckle Dimensions

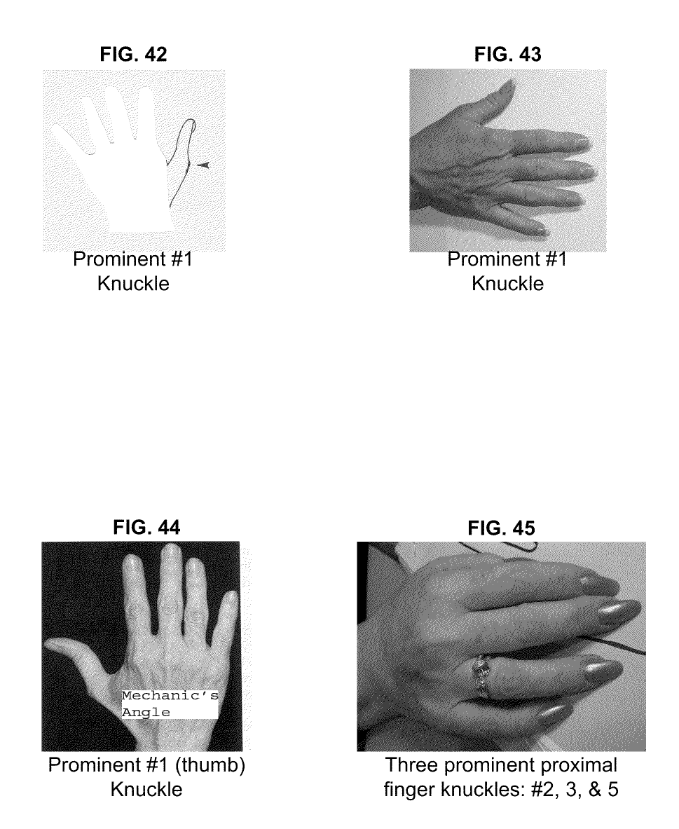

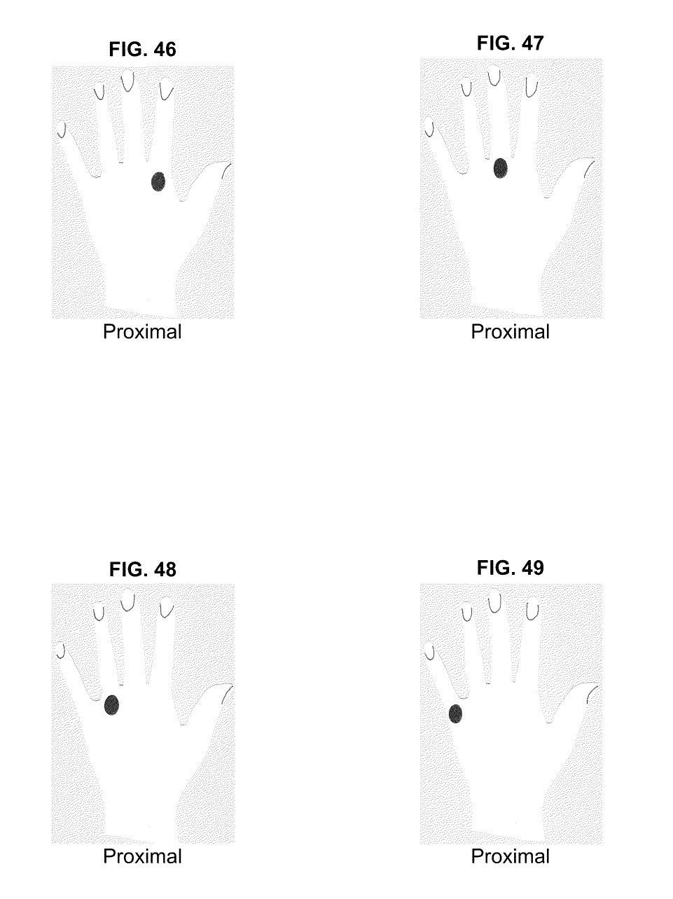

Knuckle dimensions refer to the size and shape of the knuckles on the hand. Fingers have three knuckles, the proximal, the medial, and the distal. In an embodiment, the prominence of each of these knuckles is associated with particular personality characteristics. A prominent proximal thumb knuckle is known as a mechanic's thumb. FIGS. 42, 43 and 44 provide examples of a prominent proximal thumb knuckle. A mechanic's thumb, in an embodiment, may indicate the hand is capable of almost independent action, comparable to independent intelligence, dexterity, and cleverness. A prominent proximal index finger knuckle, in an embodiment, as shown in FIGS. 45 and 46, may indicate a strong need for recognition. When found on the left hand, in an embodiment, the need for recognition is in the family sphere and when found on the right hand, in an embodiment, the need for recognition is in the work sphere. A prominent proximal middle finger knuckle, in an embodiment, as shown in FIGS. 45 and 47, may indicate a knowledge seeker.

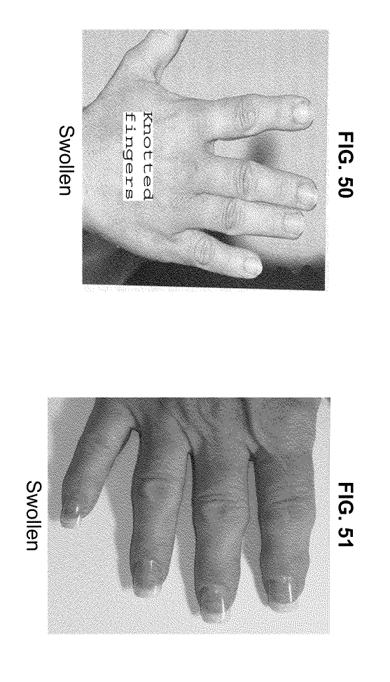

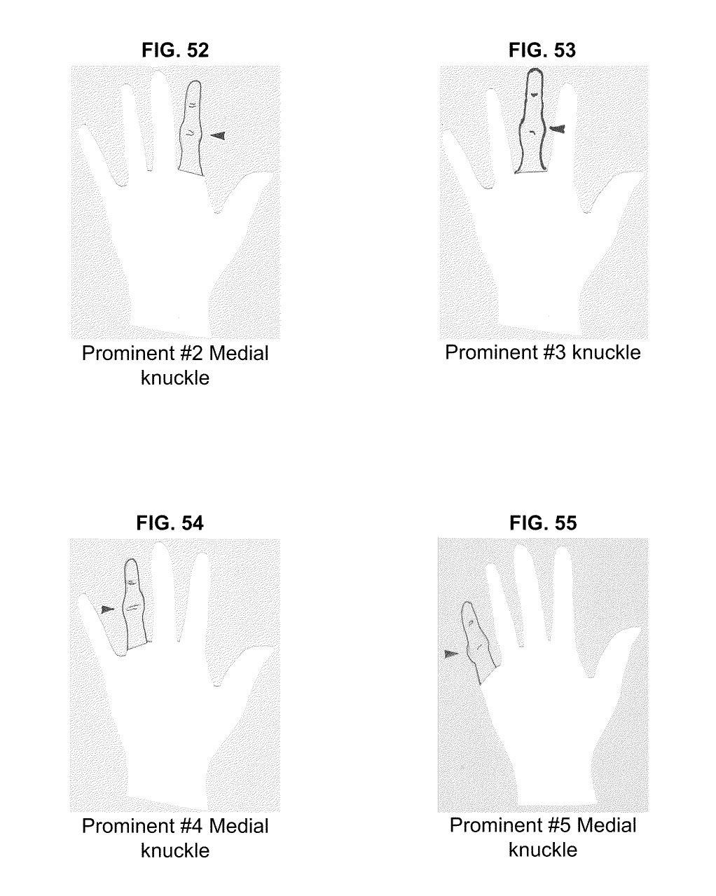

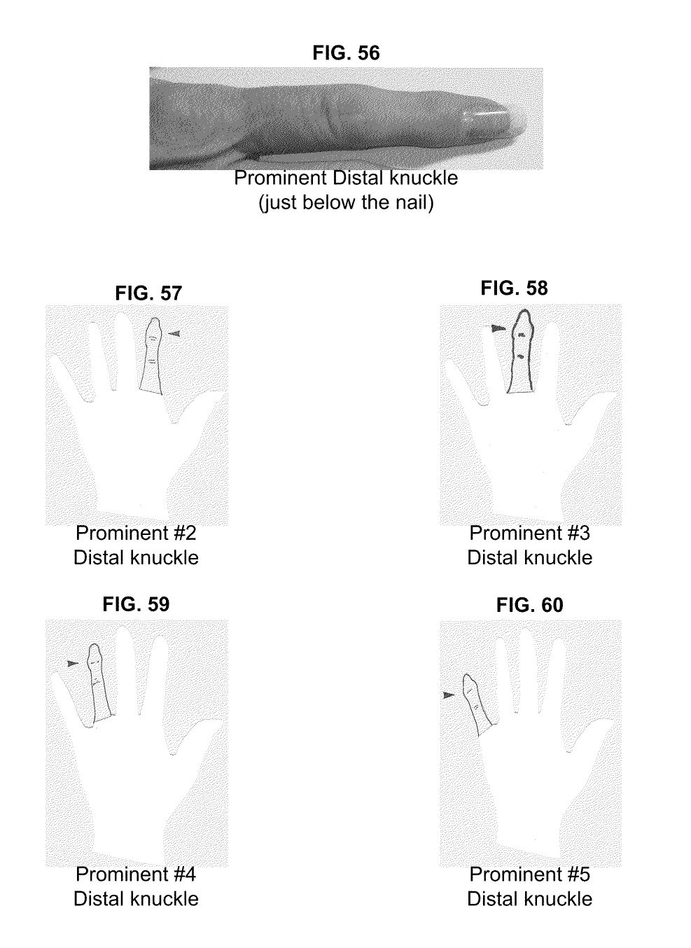

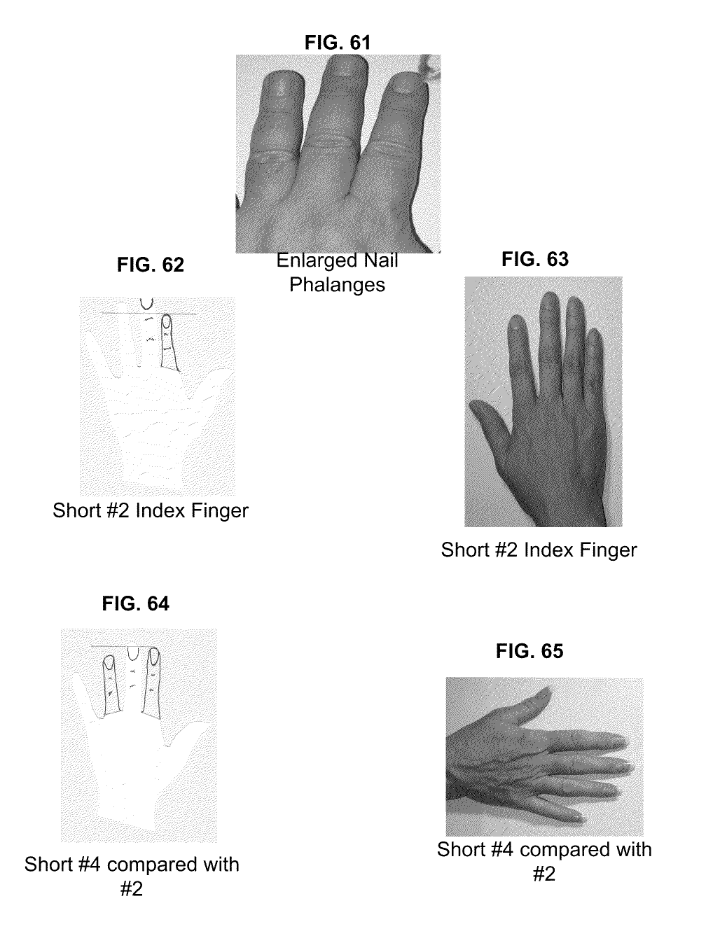

A prominent proximal ring finger knuckle, in an embodiment, as shown in FIGS. 45 and 48, may indicate a strong ambition for beauty and wealth. A prominent proximal little finger knuckle, in an embodiment, as shown in FIGS. 45 and 49, may indicate a need to garner attention to one's message. A prominent medial index finger knuckle, in an embodiment, as shown in FIGS. 50, 51, and 52, may indicate a desire to collect personal mementoes. A prominent medial middle finger knuckle, in an embodiment, as shown in FIG. 53, may indicate a desire to collect books. A prominent medial ring finger knuckle, in an embodiment, as shown in FIG. 54, may indicate a desire to collect art or other things of beauty. A prominent medial little finger knuckle, in an embodiment, as shown in FIG. 55, may indicate a desire to collect music or other forms of audio. A prominent distal index finger knuckle, in an embodiment, as shown in FIGS. 56 and 57, may indicate a tendency to debate over goals and plans. A prominent distal middle finger knuckle, in an embodiment, as shown in FIG. 58, may indicate a tendency to debate over philosophy or theology. A prominent distal ring finger knuckle, in an embodiment, as shown in FIG. 59, may indicate a tendency to debate over visual forms or beauty. A prominent distal little finger knuckle, in an embodiment, as shown in FIG. 60, may indicate a tendency to debate over music or other forms of audio. Not to be confused with prominent distal knuckles, swollen nail phalanges, in an embodiment, as shown in FIG. 61, may indicate a need to be busy, especially with one's hands.

In an embodiment, one or more knuckle dimensions, context, and any associated personality characteristics, and other similar variants, may be utilized to identify a set of personality characteristics for a subject.

Relative Finger Size

Relative finger size refers to the length of a particular finger, or a portion of the finger, with respect to the length of a one or more other fingers, or another portion of the same finger. The finger lengths measured, in an embodiment, may include the length of the index finger against the length up to the nail base of the middle finger, the length of the index finger against the length of the ring finger, the length of finger phalanges, and other similar variants.

The relative length of the index finger against the length, up to the nail base, of the middle finger, in an embodiment, as shown in FIGS. 62 and 63, can indicate a level of confidence. An index finger that is shorter than the length up to the nail base of a middle finger, in an embodiment, may indicate a desire to compensate for a lack of self confidence.

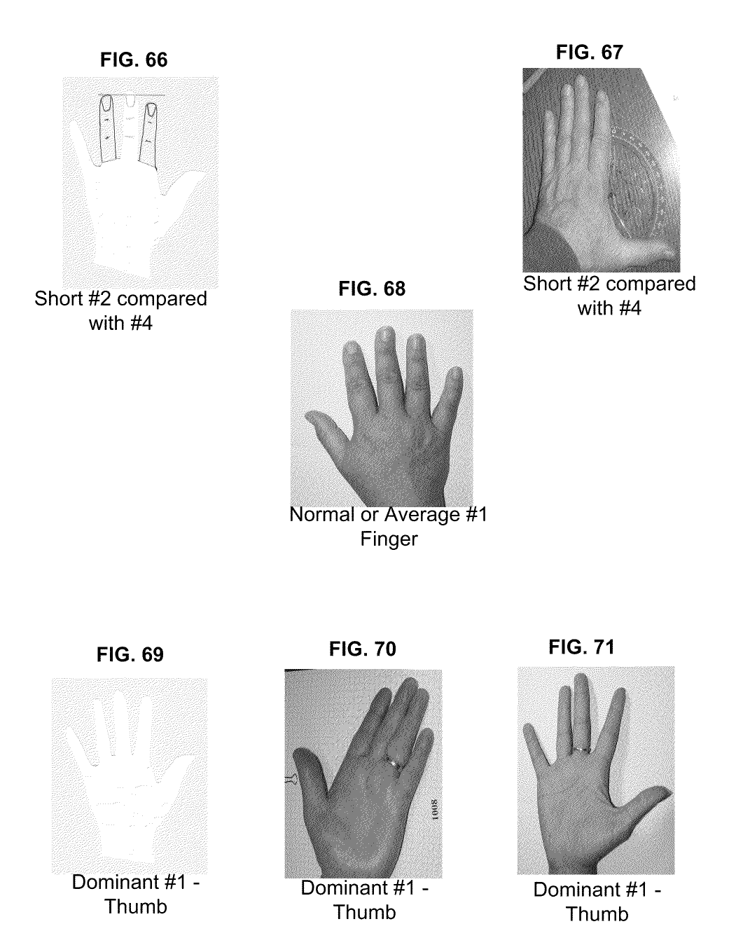

In an embodiment, The relative length of the index finger compared to the ring finger can indicate a subject's tolerance for risk. An index finger that is longer than a ring finger, in an embodiment, as shown in FIGS. 64 and 65, may indicate a cautious perspective on risk. An index finger that is shorter than a ring finger, in an embodiment, as shown in FIGS. 66 and 67, may indicate a risk taker.

The relative phalange sizes of the fingers may indicate various aspects of a subject's social activity. In an embodiment, the relative phalange sizes of the thumb may indicate a time to action. A longer distal phalange may indicate a tendency towards action without full consideration to achieve desired results, in an embodiment. A longer proximal phalange may indicate a tendency to take a longer time for consideration before action, in an embodiment. Equal relative phalange sizes may indicate a fairly coordinated consideration process that achieves results in immediate action, in an embodiment.

In an embodiment, the relative phalange sizes of the index finger may indicate a subject's emphasis on personal goals, ego, and self confidence. A longer distal phalange may indicate an emphasis on ultimate goals, in an embodiment. A longer medial phalange may indicate an emphasis on social goals, in an embodiment. A longer proximal phalange may indicate an emphasis on home and family goals, in an embodiment. Equal relative phalange sizes may indicate a balance between ultimate, social and family goals, in an embodiment.

In an embodiment, the relative phalange sizes of the middle finger may indicate a subject's emphasis on judgment and balance. A longer distal phalange may indicate an emphasis on philosophizing and a need to make or follow rules, in an embodiment. A longer medial phalange may indicate an emphasis on balance and rules in social actions, in an embodiment. A longer proximal phalange may indicate an emphasis on balance and rules in family life, in an embodiment. Equal relative phalange sizes may indicate a balance between philosophizing, a balance in social actions, and a balance in family life, in an embodiment.

In an embodiment, the relative phalange sizes of the ring finger may indicate a subject's emphasis on enthusiasm, creativity, and aesthetics. A longer distal phalange may indicate an emphasis on a need to philosophize or discuss aesthetics or matters of creative interest, in an embodiment. A longer medial phalange may indicate an emphasis on social desires to use creative energies, aesthetic interests, and enthusiasm, in an embodiment. A longer proximal phalange may indicate an emphasis on a need to direct creative energies, aesthetic interests, and enthusiasm towards home and family, in an embodiment. Equal relative phalange sizes may indicate an aesthetic, creative, and enthusiasm balance between philosophizing, social desires, and family activity, in an embodiment.

In an embodiment, the relative phalange sizes of the little finger may indicate a subject's emphasis on communication, business, math, and science. A longer distal phalange may indicate an emphasis on topics of philosophy, politics, science or theory in discussion, in an embodiment. A longer medial phalange may indicate an emphasis on friends and feelings in social communications, in an embodiment. A longer proximal phalange may indicate an emphasis on family and home in matters of communication and business, in an embodiment. A shorter medial phalange may indicate a lack of interest in social conversation and communication, little time for “small talk,” in an embodiment. Equal relative phalange sizes may indicate a balance between philosophy, social, and family communication, in an embodiment.

In an embodiment, one or more relative finger lengths, context, and any associated personality characteristics, and other similar variants, may be utilized to identify a set of personality characteristics for a subject.

Finger Dimensions

Finger dimensions refer to the size and shape of the fingers of the hand. In an embodiment, various aspects of a particular finger, for instance length, size, or shape, may be associated with various personality characteristics.

In an embodiment, Thumb size may be associated with a controlling personality characteristic. FIG. 68 shows an example of an average size thumb. An above average size thumb, in an embodiment, as shown in FIGS. 69, 70, and 71, may indicate a domineering personality characteristic.

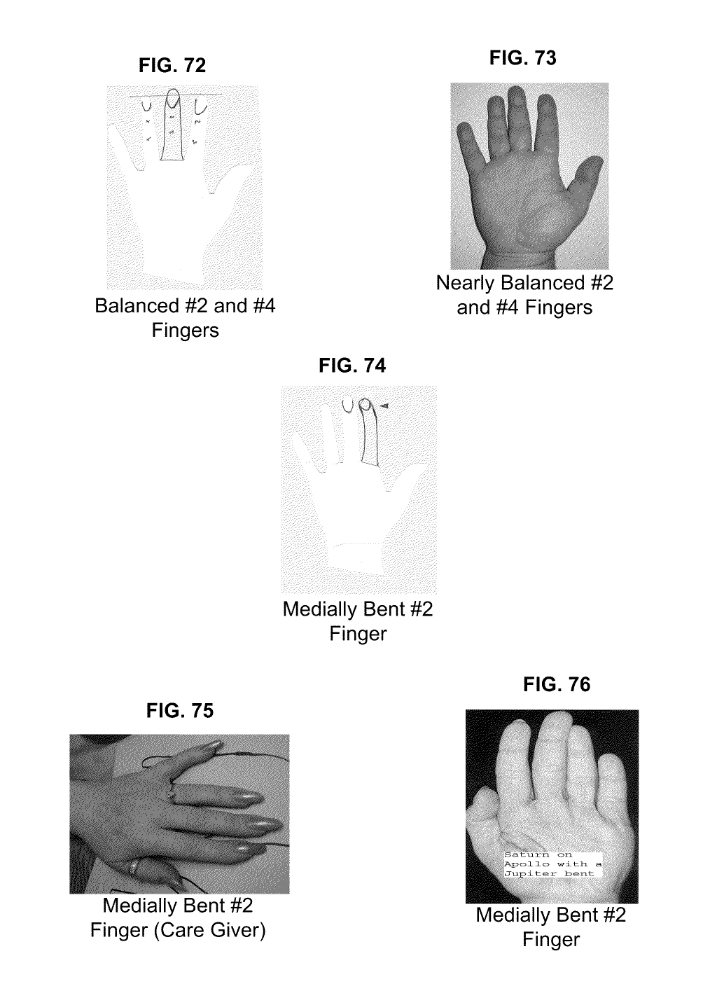

In an embodiment, relatively equal index and ring fingers may indicate a balanced personality characteristic. An index finger and ring finger that are nearly equal in length and overall size, in an embodiment, as shown in FIGS. 72 and 73, may indicate a tendency to avoid risk but not to be overly cautious.

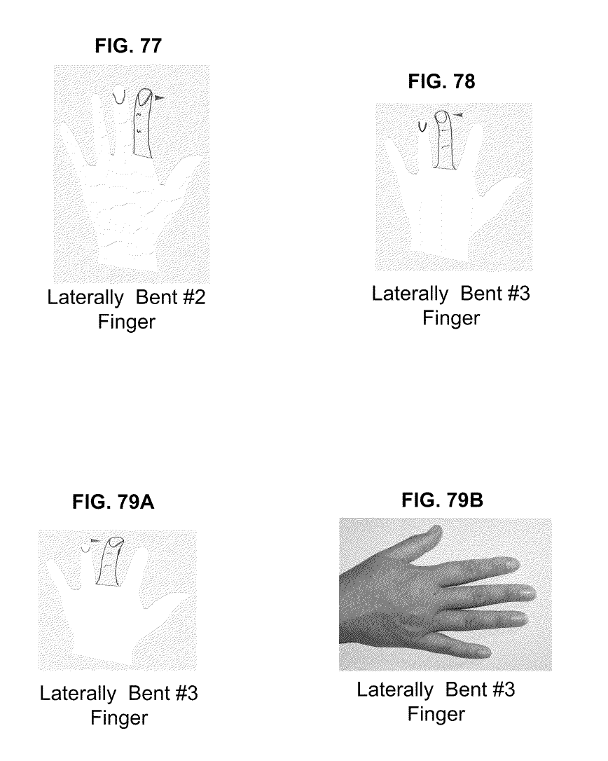

In an embodiment, the shape of the index finger may indicate a subject's generosity. A medially bent index finger, in an embodiment, as shown in FIGS. 74, 75, and 76, may indicate a willingness to put others first. A laterally bent index finger, in an embodiment, as shown in FIG. 77, may indicate a tendency to put oneself first.

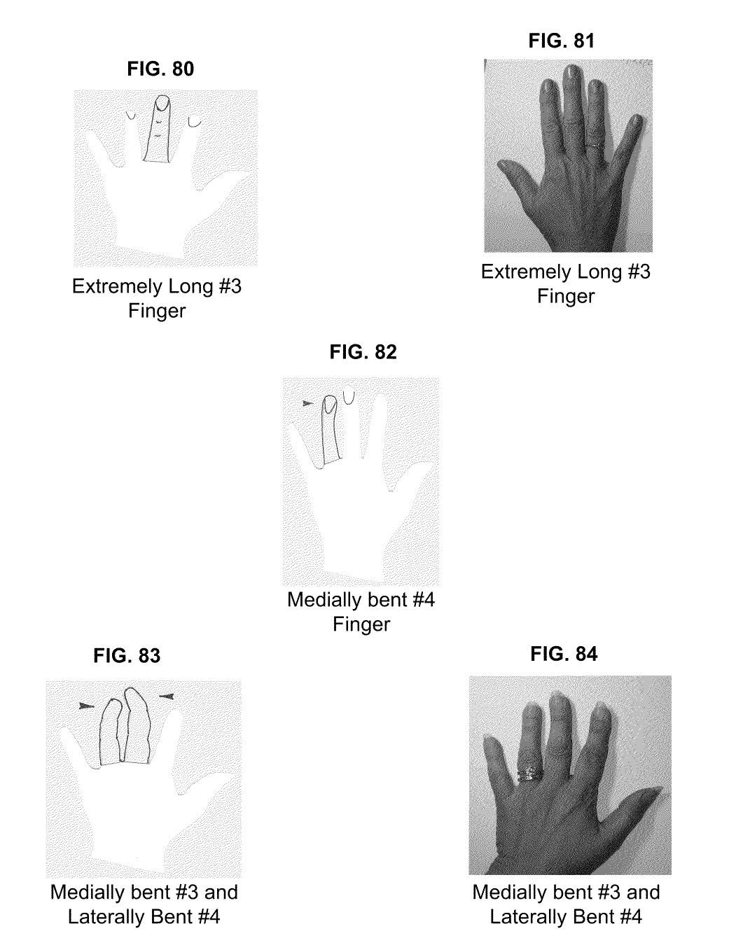

In an embodiment, the shape of the middle finger can indicate tendencies in assigning fault. A medially bent middle finger, in an embodiment, as shown in FIGS. 78, 79A and 76, may indicate a self-critic. A laterally bent middle finger, in an embodiment, as shown in FIG. 79B, may indicate a critic of others. And a middle finger that is extra long, in an embodiment, as shown in FIGS. 80 and 81, may indicate a hypercritic.

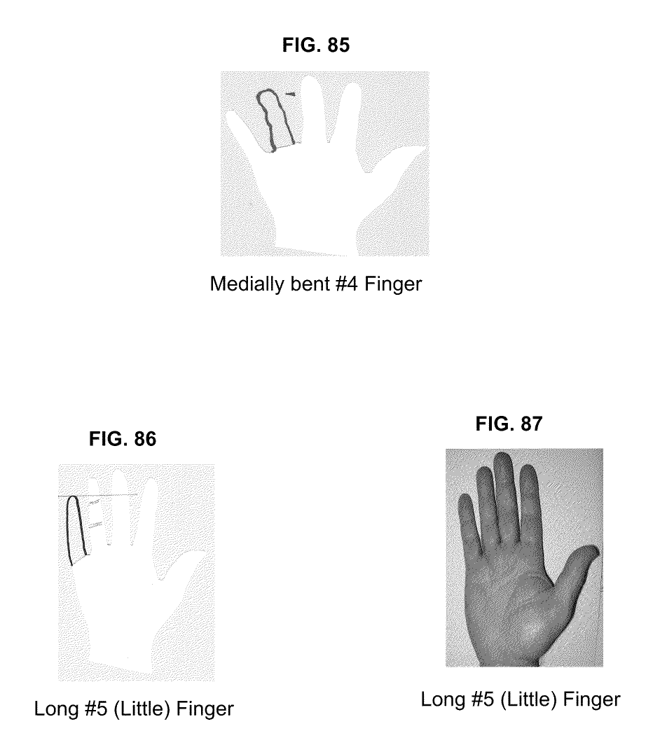

In an embodiment, the shape of the ring finger can indicate a willingness to share. A laterally bent ring finger, in an embodiment, as shown in FIG. 82, may indicate a willingness to share one's work, sometimes through performance. A medially bent middle finger and a laterally bent ring finger, in an embodiment, as shown in FIGS. 83 and 84, may indicate an internal conflict between a desire to perform but a lack of confidence. And a laterally bent ring finger, in an embodiment, as shown in FIG. 85, may indicate a tendency not to share one's work.

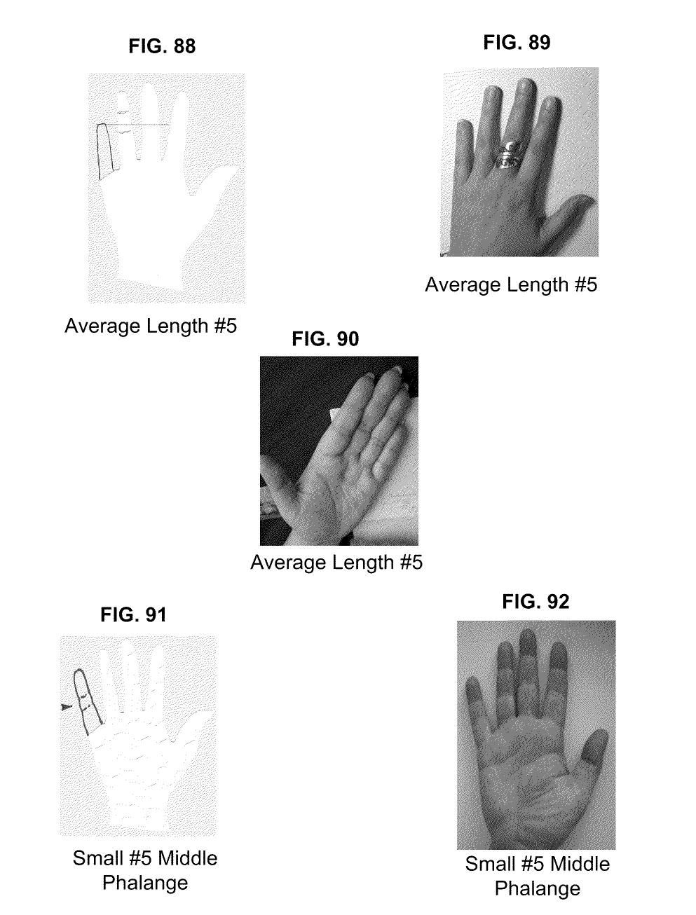

In an embodiment, the length of the little finger may indicate an intelligence level. A short little finger, in an embodiment, as shown in FIGS. 86 and 87, may indicate high intelligence. An average length little finger, in an embodiment, as shown in FIGS. 88, 89, and 90, may indicate adequate intelligence.

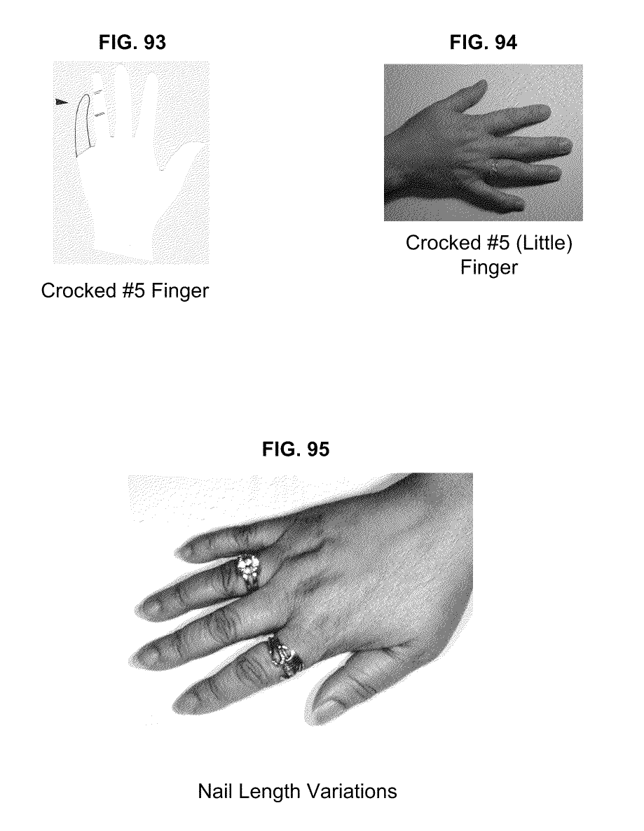

In an embodiment, the shape of the little finger can indicate a subject's tendencies in dealing with others. A little finger with a small medial phalange relative to the distal and proximal phalanges, in an embodiment, as shown in FIGS. 91 and 92, may indicate an intolerance for small talk. A little finger that extends in a crooked direction, in an embodiment, as shown in FIGS. 93 and 94, may indicate a tendency to embellish the truth.

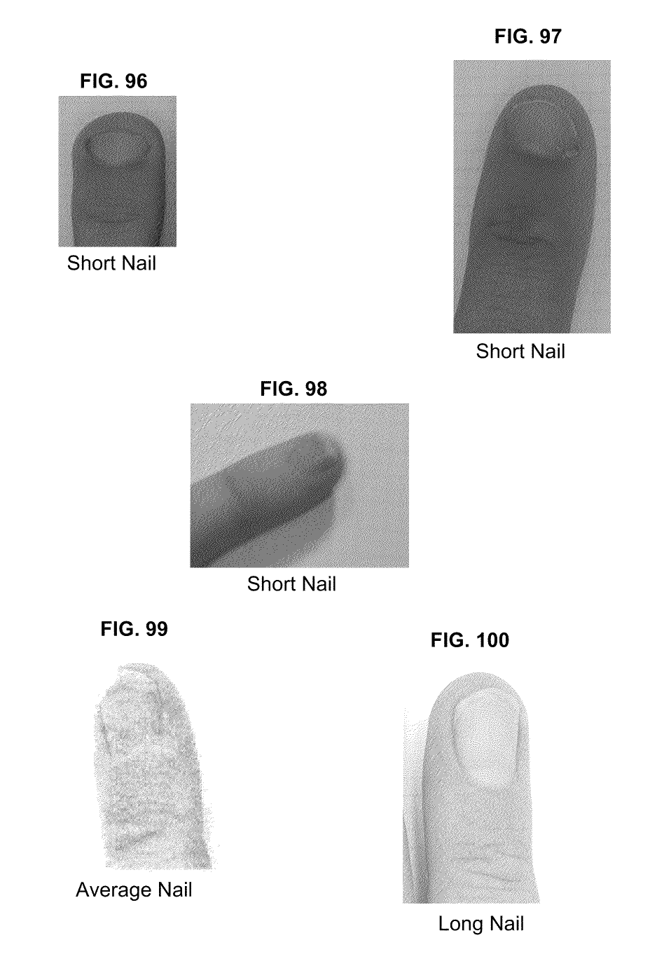

In an embodiment, the length of the nail of a finger can indicate a quickness of the mind. A short nail, in an embodiment, as shown in FIGS. 96, 97, and 98, may indicate a quick mind. An average length nail, in an embodiment, as shown in FIG. 99, may indicate average speed of the mind. And a long nail, in an embodiment, as shown in FIG. 100, may indicate a deliberate mind. In an embodiment, the length of nails can differ for each finger, as shown in FIG. 95, however the mismatch processing later described provides an example resolution for mismatched personality characteristics.

In an embodiment, one or more finger dimensions, context, and any associated personality characteristics, and other similar variants, may be utilized to identify a set of personality characteristics for a subject.

Computing Environment

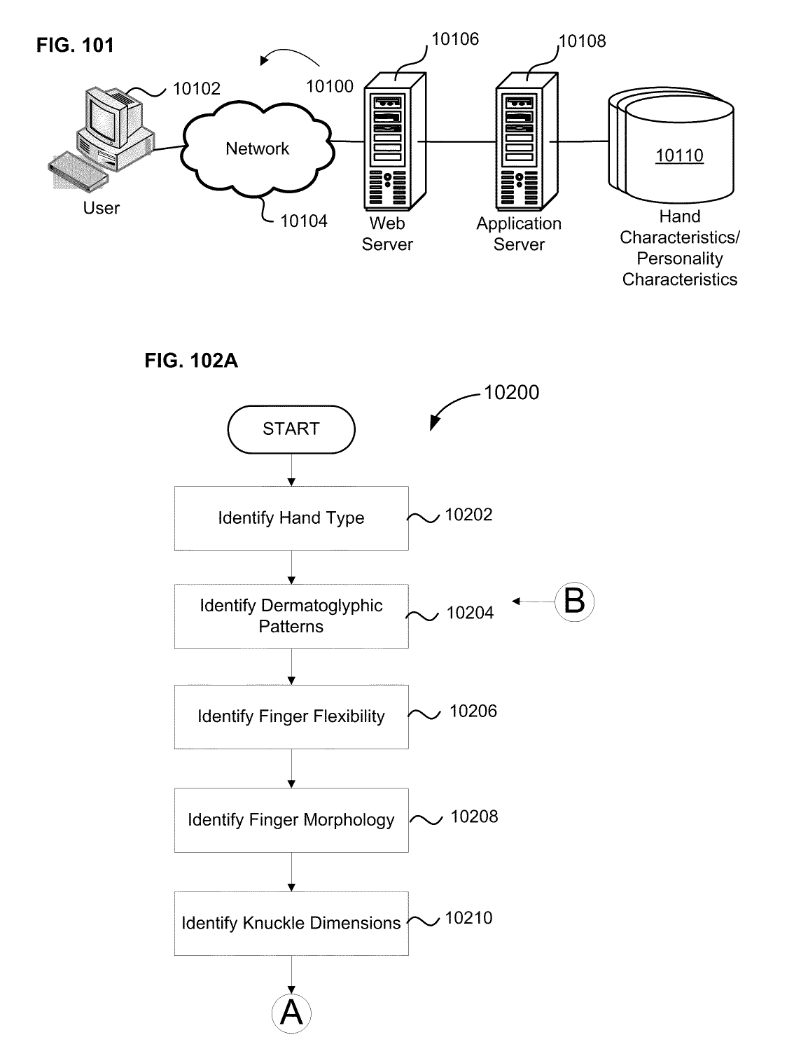

A computing environment is utilized to practice an embodiment. FIG. 101 illustrates an example of an environment 10100 for implementing aspects in accordance with various embodiments. As will be appreciated, although a Web-based environment is used for purposes of explanation, different environments may be used, as appropriate, to implement various embodiments. The environment 10100 includes an electronic client device 10102, which can include any appropriate device operable to send and receive requests, messages, or information over an appropriate network 10104 and convey information back to a user of the device. Examples of such client devices include personal computers, cell phones, handheld messaging devices, laptop computers, set-top boxes, personal data assistants, electronic book readers, and the like. The network can include any appropriate network, including an intranet, the Internet, a cellular network, a local area network, or any other such network or combination thereof. Components used for such a system can depend at least in part upon the type of network and/or environment selected. Protocols and components for communicating via such a network are well known and will not be discussed herein in detail. Communication over the network can be enabled by wired or wireless connections, and combinations thereof. In this example, the network includes the Internet, and the environment includes a Web server 10106 for receiving requests and serving content in response thereto, although for other networks an alternative device serving a similar purpose could be used as would be apparent to one of ordinary skill in the art.

The computer system 10100 described above, or variations thereof, or multiple computer systems may be utilized in order to implement various embodiments of the invention. In addition, executable instructions for practicing the invention may be collectively stored on one or more computer-readable storage media. One or more computer systems that may include one or more processors may collectively execute the instructions.

The illustrative environment includes at least one application server 10108 and a data store 10110. It should be understood that there can be several application servers, layers, or other elements, processes, or components, which may be chained or otherwise configured, which can interact to perform tasks such as obtaining data from an appropriate data store. As used herein the term “data store” refers to any device or combination of devices capable of storing, accessing, and retrieving data, which may include any combination and number of data servers, databases, data storage devices, and data storage media, in any standard, distributed, or clustered environment. The application server can include any appropriate hardware and software for integrating with the data store as needed to execute aspects of one or more applications for the client device, handling a majority of the data access and business logic for an application. The application server provides access control services in cooperation with the data store, and is able to generate content such as text, graphics, audio, and/or video to be transferred to the user, which may be served to the user by the Web server in the form of HTML, XML, or another appropriate structured language in this example. The handling of all requests and responses, as well as the delivery of content between the client device 10102 and the application server 10108, can be handled by the Web server. It should be understood that the Web and application servers are not required and are merely example components, as structured code discussed herein can be executed on any appropriate device or host machine as discussed elsewhere herein.

The data store 10110 can include several separate data tables, databases, or other data storage mechanisms and media for storing data relating to a particular aspect. For example, the data store illustrated includes mechanisms for storing personality characteristic data associated with hand characteristic data, which can be used to serve content for the production side. It should be understood that there can be many other aspects that may need to be stored in the data store, such as for page image information and to access right information, which can be stored in any of the above listed mechanisms as appropriate or in additional mechanisms in the data store 10110. The data store 10110 is operable, through logic associated therewith, to receive instructions from the application server 10108 and obtain, update, or otherwise process data in response thereto.

Each server typically will include an operating system that provides executable program instructions for the general administration and operation of that server, and typically will include a computer-readable medium storing instructions that, when executed by a processor of the server, allow the server to perform its intended functions. Suitable implementations for the operating system and general functionality of the servers are known or commercially available, and are readily implemented by persons having ordinary skill in the art, particularly in light of the disclosure herein.

The environment in one embodiment is a distributed computing environment utilizing several computer systems and components that are interconnected via communication links, using one or more computer networks or direct connections. However, it will be appreciated by those of ordinary skill in the art that such a system could operate equally well in a system having fewer or a greater number of components than are illustrated in FIG. 101. Thus, the depiction of the system 10100 in FIG. 101 should be taken as being illustrative in nature, and not limiting to the scope of the disclosure.

User Interface

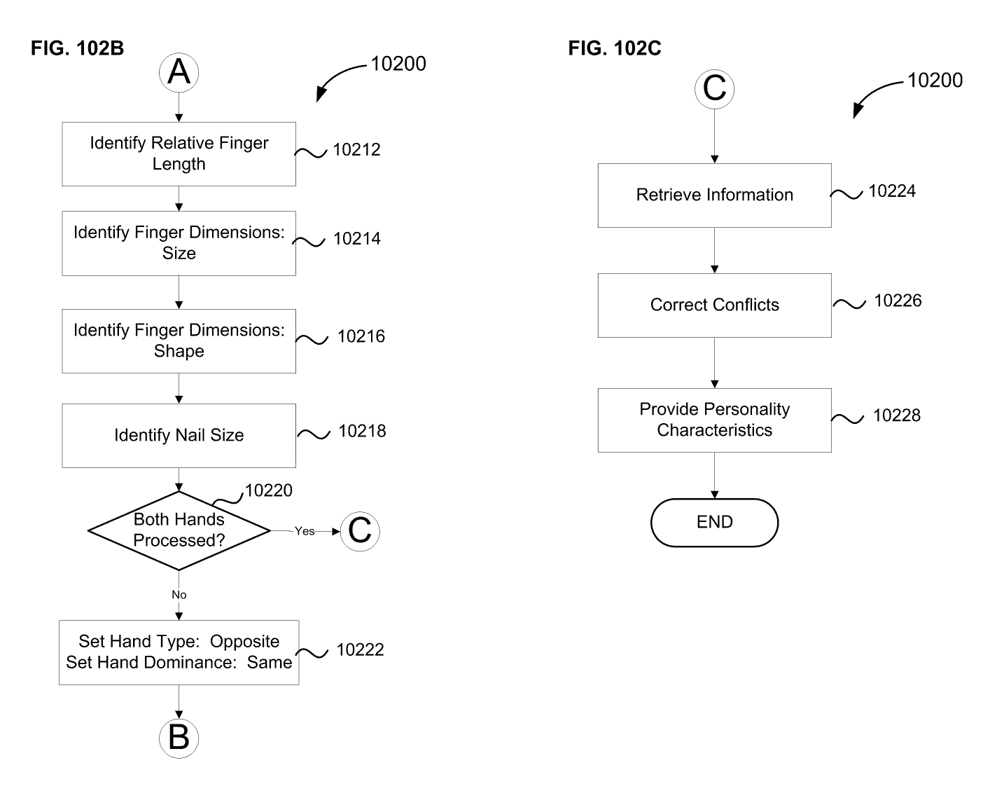

The following will describe step-by-step illustrative embodiments of processes that may be performed, at least in part, on a computing environment such as the one described above. In particular, FIGS. 102A-C depict a flowchart that show illustrative examples of processes that may be used together in a process 10200 for providing a personality inventory. The processes shown and described in the present disclosure, combinations and/or variations thereof may be performed under the control of one or more computer systems that are configured with executable instructions. Executable instructions may be collectively stored on one or more computer-readable storage media.

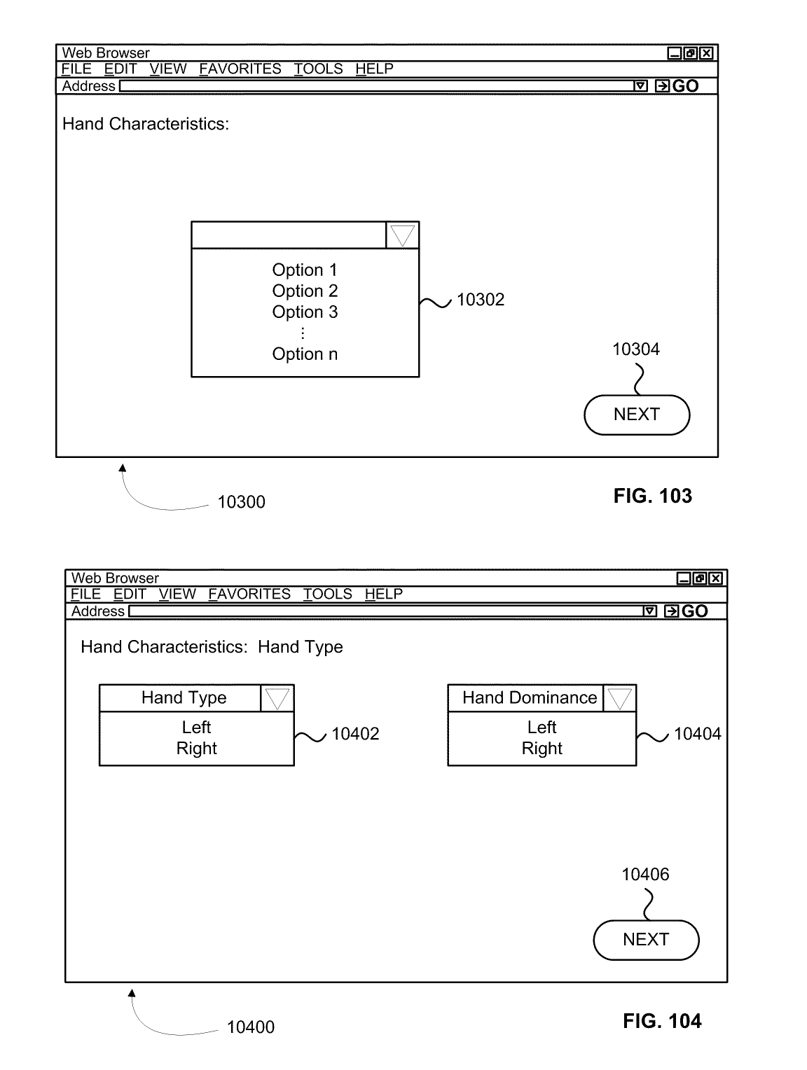

The flowchart includes, in addition to several other steps, a series of steps identifying hand characteristics. Identifying hand characteristics can be performed in various ways. For instance, FIG. 103 describes a web browser equipped with code that provides for a user interface 10300, in accordance with an embodiment. The user interface may contain text forms, such as drop-down menu 10302, for receiving hand characteristic information from the user and a “Next” button 10304 for progressing. The user interface may further utilize text-box forms, radio forms, various menus forms, and any other suitable web technologies, such as JavaScript, Asynchronous JavaScript and XML, HTML, XHTML, Cascading Style Sheets, Java, Java Server Pages, JavaBeans, J2EE, Active Server Pages, .NET Framework, PHP, and any other suitable technology, and any combination. The user interface may comprise a Hypertext Protocol webpage, a portal, an applet, or any other suitable technology, and any combination. The sample user interface is only a single embodiment and a number of other designs or technologies and/or ways of obtaining information about hand characteristics may be implemented. For example, while the sample interface utilizes dropdown menus, other interface elements may be used alternatively or in addition to dropdown menus. For instance, checkboxes and/or radio buttons may be used.

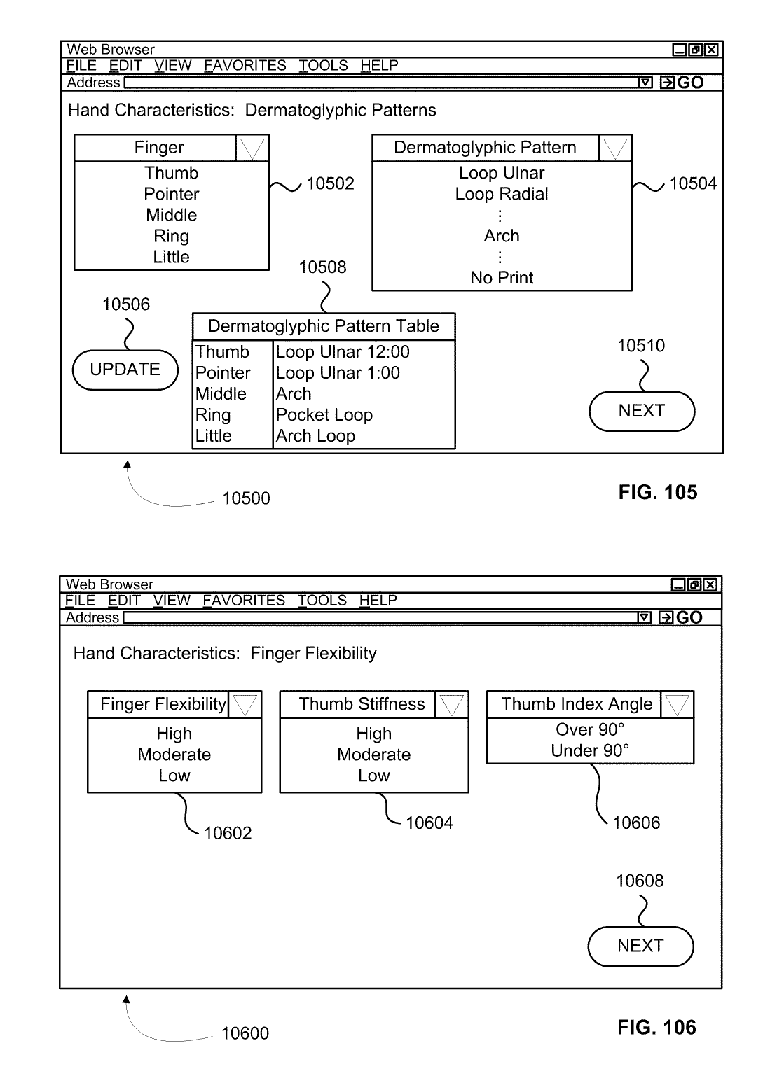

Returning to FIG. 102A, in one embodiment, Identifying Hand Type 10202 is the first step of the process 10200. Identification of the hand type includes identifying, in this example, whether the hand is right or left, respective to the subject, and whether the subject is right or left hand dominant. FIG. 104 describes a Hand Type User Interface page 10400 of the browser shown in FIG. 103, in accordance with an embodiment. The Hand Type page contains drop-down menus 10402 and 10404. A user may specify Hand Type (left or right) with menu 10402 and Hand Dominance (left or right) with menu 10404. Once the Hand Type and Hand Dominance are specified, in an embodiment, the user clicks (i.e., selects using appropriate user input) a “Next” button 10406 to continue. Responsive to clicking the “Next” button 10406, the browser may send a request for another page from a web server. The request may include information identifying the selections made by the user. Responsive to the request, the web server may send an appropriate page to a device of the user on which the browser resides. FIG. 105 is an example of such a page that may be sent by a web server in response to clicking on the “Next” button 10406. In another embodiment, Asynchronous JavaScript and XML techniques are used in order to present new menus to the user responsive to selections being made.

Referring again to FIG. 102A, in one embodiment, Identifying Dermatoglyphic Patterns 10204 is the next step of the process 10200. Identification may include identifying, in this example, dermatoglyphic patterns for one or more fingers on the specified hand. FIG. 105, in an accordance with an embodiment, describes the Dermatoglyphic Patterns User Interface page 10500, which may be presented responsive to a user having submitted selections in connection with the Hand Type Interface Page 10400, described above.

In an embodiment, The Dermatoglyphic Patterns page contains drop-down menus 10502 and 10504, button 10506, and table 10508. The user may first, for example, specify a finger using menu 10502. Next, the user may specify a dermatoglyphic pattern, for instance an ulnar loop 12:00, radial loop 1:00, tented arch, elongated whorl, etc, using menu 10504. Once these are specified, the user may click an “Update Table” button 10506. The “Update Table” button 10506, in an embodiment, configures the “Dermatoglyphic Pattern Table” 10508 with hand characteristic information. The “Update Table” button 10506, once clicked by the user, may add the finger and dermatoglyphic pattern information specified through menus 10502 and 10504 to the “Dermatoglyphic Pattern Table” 10508, in accordance with an embodiment. Upon updating the “Dermatoglyphic Pattern Table” 10508, in an embodiment, the browser may clear the specified finger and dermatoglyphic pattern information from menus 10502 and 10504 so that the user may specify a next finger and a next dermatoglyphic pattern. In an embodiment, the user may repeat this process until each finger of the specified hand has been added to the table.

The Dermatoglyphic Patterns User Interface page 10500 described above is only a sample embodiment for receiving dermatoglyphic pattern information and other techniques may be utilized. In an embodiment, once the table is complete, the user clicks “Next” 10510 to continue. Responsive to clicking the “Next” button 10510, the browser may send a request for another page from a web server. The request may include information identifying the selections made by the user. Responsive to the request, the web server may send an appropriate page to a device of the user on which the browser resides. FIG. 106 is an example of such a page that may be sent by a web server in response to clicking on the “Next” button 10510.

Referring again to FIG. 102A, in one embodiment, Identifying Finger Flexibility 10206 is the next step of the process 10200. Identification may include identifying, in this example, finger flexibility, thumb stiffness, and the angle between the thumb and index fingers for the specified hand. FIG. 106, in accordance with an embodiment, describes the Finger Flexibility User Interface page 10600, which may be presented responsive to a user having submitted selections in connection with the Dermatoglyphic Pattern User Interface Page 10500, described above. The Finger Flexibility page may contain drop-down menus 10602, 10604 and 10606. In an embodiment, the user may specify Finger flexibility (high, moderate, or low) with menu 10602, Thumb stiffness (high, moderate, or low) with menu 10604 and Thumb Index Angle (over 90° or under 90°) with menu 10606.

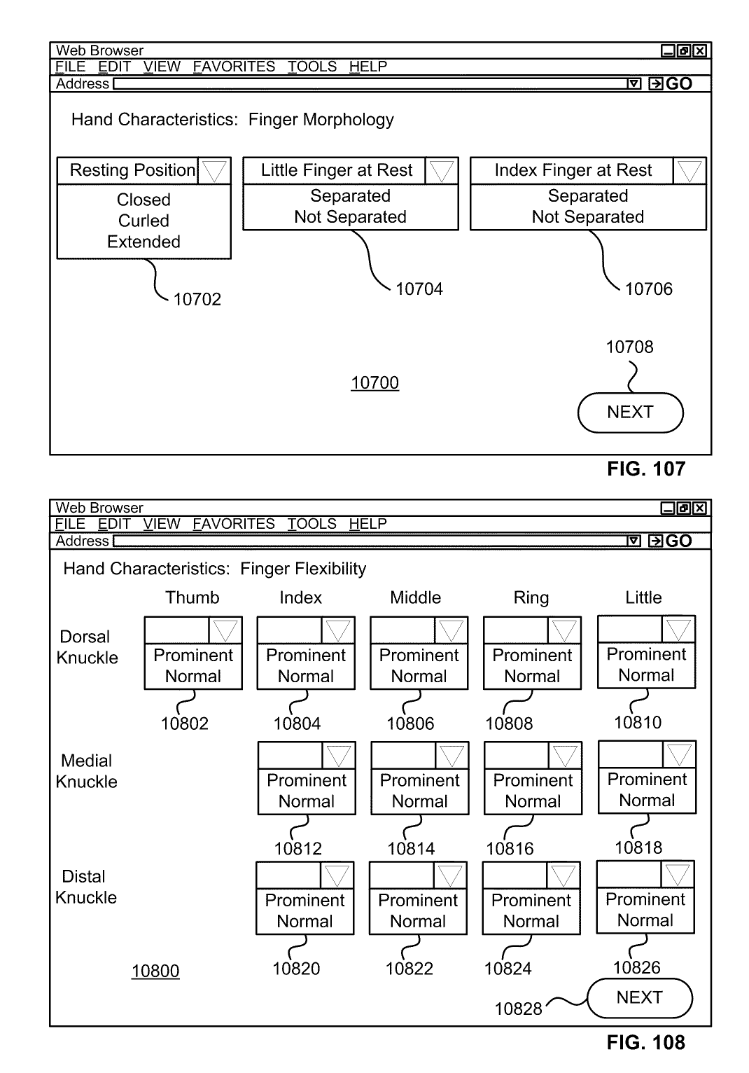

The Finger Flexibility User Interface page 10600 described above is only an sample embodiment for receiving finger flexibility information and other techniques may be utilized. In an embodiment, once each of the drop-down menus has been specified, the user clicks “Next” 10608 to continue. Responsive to clicking the “Next” button 10608, the browser may send a request for another page from a web server. The request may include information identifying the selections made by the user. Responsive to the request, the web server may send an appropriate page to a device of the user on which the browser resides. FIG. 107 is an example of such a page that may be sent by a web server in response to clicking on the “Next” button 10608.

Referring again to FIG. 102A, in one embodiment, Identifying Finger Morphology 10208 is the next step of the process 10200. Identification may include identifying, for this example, finger tendencies at resting position, the little finger at rest, and the index finger at rest for the specified hand. FIG. 107, in accordance with an embodiment, describes the Finger Morphology User Interface page 10700, which may be presented responsive to a user having submitted selections in connection with the Finger Flexibility User Interface Page 10600, described above. The Finger Morphology page may contain drop-down menus 10702, 10704 and 10706. I an embodiment, the user may specify Finger Resting Position (closed, curled, or extended) with menu 10702, Little Finger At Rest (separated or not separated) with menu 10704 and Index Finger At Rest (separated or not separated) with menu 10706.

The Finger Morphology User Interface page 10700 described above is only an sample embodiment for receiving finger morphology information and other techniques may be utilized. In an embodiment, once each of the drop-down menus has been specified, the user may click “Next” 10708 to continue. Responsive to clicking the “Next” button 10708, the browser may send a request for another page from a web server. The request may include information identifying the selections made by the user. Responsive to the request, the web server may send an appropriate page to a device of the user on which the browser resides. FIG. 108 is an example of such a page that may be sent by a web server in response to clicking on the “Next” button 10708.

Referring again to FIG. 102A, in one embodiment, Identifying Knuckle Dimensions 10210 is the next step of the process 10200. Identification may include identifying, for this example, knuckle dimensions for the dorsal, medial, and distal knuckles on each finger of the specified hand. FIG. 108, in accordance with an embodiment, describes the Knuckle Dimensions User Interface page 10800, which may be presented responsive to a user having submitted selections in connection with the Finger Morphology User Interface Page 10700, described above. The Knuckle Dimensions page may contain drop-down menus 10802, 10804, 10806, 10808, 10810, 10812, 10814, 10816, 10818, 10820, 10822, 10824, and 10826. In an embodiment, the user may specify Dorsal Knuckle dimensions (prominent or normal) for each finger with menus 10802, 10804, 10806, 10808, and 10810, Medial Knuckle dimensions (prominent or normal) for each finger with menus 10812, 10814, 10816, and 10818, and Distal Knuckle dimensions (prominent or normal) for each finger with menus 10820, 10822, 10824, and 10826.

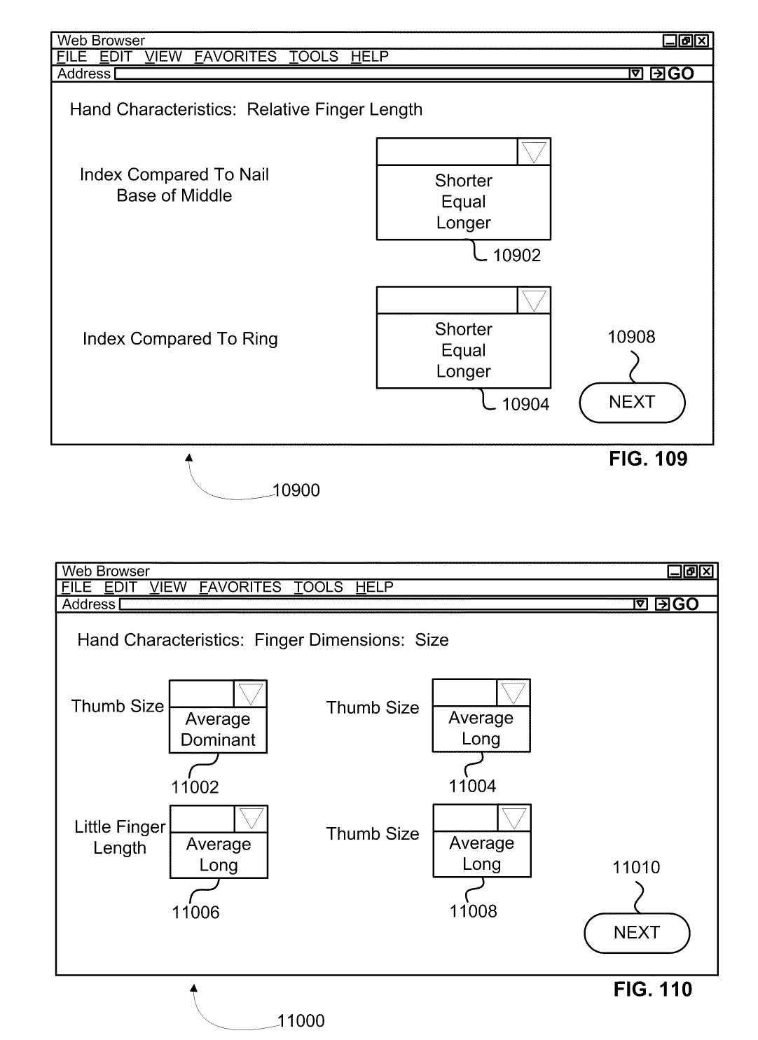

The Knuckles Dimensions User Interface page 10800 described above is only an sample embodiment for receiving finger morphology information and other techniques and technologies, such as menus utilizing dynamic Asynchronous JavaScript and XML (AJAX) technologies, may be utilized. In an embodiment, once each of the drop-down menus has been specified, the user may click “Next” 10828 to continue. Responsive to clicking the “Next” button 10828, the browser may send a request for another page from a web server. The request may include information identifying the selections made by the user. Responsive to the request, the web server may send an appropriate page to a device of the user on which the browser resides. FIG. 109 is an example of such a page that may be sent by a web server in response to clicking on the “Next” button 10828.

Referring to FIG. 102B, in one embodiment, Identifying Relative Finger Length 10212 is the next step of the process 10200. Identification may include identifying, for this example, the index finger compared to the nail base of the middle finger and the index finger compared to the ring finger for the specified hand. FIG. 109, in accordance with an embodiment, describes the Relative Finger Length User Interface page 10900, which may be presented responsive to a user having submitted selections in connection with the Knuckles Dimensions User Interface Page 10800, described above. The Relative Finger Length page may contain drop-down menus 10902 and 10904. In an embodiment, the user may specify Index Compared To Nail Base Of Middle (shorter, equal or longer) with menu 10902 and Index Compared To Ring (shorter, equal or longer) with menu 10904.

The Relative Finger Length User Interface page 10900 described above is only an sample embodiment for receiving relative finger length information and other techniques may be utilized. In an embodiment, once each of the drop-down menus has been specified, the user may click “Next” 10906 to continue. Responsive to clicking the “Next” button 10906, the browser may send a request for another page from a web server. The request may include information identifying the selections made by the user. Responsive to the request, the web server may send an appropriate page to a device of the user on which the browser resides. FIG. 110 is an example of such a page that may be sent by a web server in response to clicking on the “Next” button 10906.

Referring to FIG. 102B, in one embodiment, Identifying Finger Dimensions: Size 10214 is the next step of the process 10200. Identification may include identifying, for this example, the thumb size, the middle finger length, the little finger length, and the little finger medial phalange size for the specified hand. FIG. 110, in accordance with an embodiment, describes the Finger Dimensions: Size User Interface page 11000, which may be presented responsive to a user having submitted selections in connection with the Relative Finger Length User Interface Page 10900, described above. The Finger Dimensions: Size page may contain drop-down menus 11002, 11004, 11006, and 11008. In an embodiment, the user may specify Thumb Size (average or dominant) with menu 11002, Middle Finger Length (average or long) with menu 11004, Little Finger Length (average or long) with menu 11006, and Little Finger Medial Phalange Size (average or small) with menu 11008.

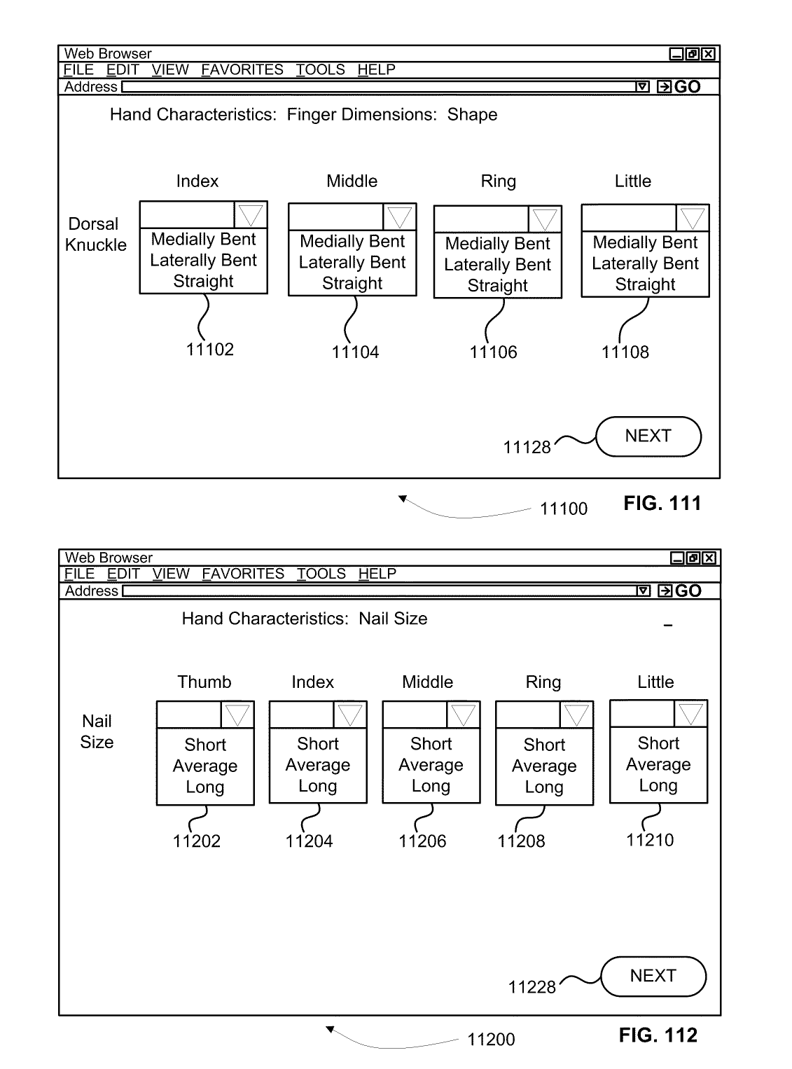

The Finger Dimensions: Size User Interface page 11000 described above is only an sample embodiment for receiving finger dimensions size information and other techniques may be utilized. In an embodiment, once each of the drop-down menus has been specified, the user may click “Next” 11010 to continue. Responsive to clicking the “Next” button 11010, the browser may send a request for another page from a web server. The request may include information identifying the selections made by the user. Responsive to the request, the web server may send an appropriate page to a device of the user on which the browser resides. FIG. 111 is an example of such a page that may be sent by a web server in response to clicking on the “Next” button 11010.

Referring again to FIG. 102B, in one embodiment, Identifying Finger Dimensions: Shape 10216 is the next step of the process 10200. Identification may include identifying, for this example, finger alignment for the index, middle, ring, and little fingers of the specified hand. FIG. 111, in accordance with an embodiment, describes the Finger Dimensions: Shape User Interface page 11100, which may be presented responsive to a user having submitted selections in connection with the Finger Dimensions: Size User Interface Page 11000, described above. The Finger Dimensions: Shape page may contain drop-down menus 11102, 11104, 11106, and 11108. In an embodiment, the user may specify Finger Alignment for the index, middle and ring fingers (medially bent, laterally bent, or straight) with menus 11102, 11104, and 11106 and Finger Alignment for the little finger (crooked or straight) with menu 11108.

The Finger Dimensions: Shape User Interface page 11100 described above is only an sample embodiment for receiving finger dimensions shape information and other techniques may be utilized. In an embodiment, once each of the drop-down menus has been specified, the user clicks “Next” 11110 to continue. Responsive to clicking the “Next” button 11110, the browser may send a request for another page from a web server. The request may include information identifying the selections made by the user. Responsive to the request, the web server may send an appropriate page to a device of the user on which the browser resides. FIG. 112 is an example of such a page that may be sent by a web server in response to clicking on the “Next” button 11110.

Referring again to FIG. 102B, in one embodiment, Identifying Nail Size 10218 is the next step of the process 10200. Identification may include identifying, for this example, nail sizes for the fingers of the specified hand. FIG. 112, in accordance with an embodiment, describes the Nail Size User Interface page 11200, which may be presented responsive to a user having submitted selections in connection with the Finger Dimensions: Shape User Interface Page 11100, described above. The Nail Size page may contain drop down menus 11202, 11204, 11206, 11208 and 11210. In an embodiment, the user may specify a Nail Size for each finger (short, average, or long) with menus 11202, 11204, 11206, 11208 and 11210.

The Nail Size User Interface page 11200 described above is only an sample embodiment for receiving nail size information and other techniques, such as dynamic Asynchronous JavaScript and XML menus, may be utilized. In an embodiment, once each of the drop-down menus has been specified, the user clicks “Next” 11212 to continue. Responsive to clicking the “Next” button 11212, the browser may send a request for another page from a web server. The request may include information identifying the selections made by the user. Responsive to the request, the web server may send an appropriate page to a device of the user on which the browser resides.

Referring again to FIG. 102B, in one embodiment, determining whether both hands have been processed 10220 is the next step of process 10200. If both hands have not been processed, in accordance with an embodiment, the process may proceed to the setting step 10222. The setting step may include setting the hand type to the opposite (left to right or right to left) and setting the hand dominance to the same (left or right). From the setting step 10222, in accordance with an embodiment, the process repeats starting from the Identifying Dermatoglyphic Patterns step 10204 of FIG. 102A, for the opposite hand. The repeated process may implement a user interface, as described above, or any other suitable means for receiving hand characteristic information.

Referring to FIG. 102C, in one embodiment, if both hands have been processed, Retrieving Information 10224 is the next step of the process 10200. Retrieving may include, for example, querying a data store with hand characteristic information and receiving associated personality characteristic information.

Referring to FIG. 102C, in one embodiment, Correcting Conflicts 10226 is the next step of the process 10200. Correcting may include, for this example, determining whether any mismatched personality characteristics exist, and correcting any mismatch. For instance, the hand characteristic information may comprise a variety of nail lengths for each finger on each hand. Since different nail lengths indicate a degree of mental quickness, different nails lengths may cause a conflict in the associated personality characteristics. In one embodiment, the process utilizes a context of the hand characteristics to provide a resolution. In accordance with an embodiment, an example mismatch may be a short nail on the little finger, but a long nail on the thumb. Exemplary corrected personality characteristics for this mismatch may include a subject that speaks quickly, since a short nail may indicate a quick mind and hand characteristics found on the pinky may be associated with communication, but deliberates on actions, since a long nail may indicate deliberation and hand characteristics found on the thumb may be associated with actions. This sample mismatch and correction is a mere example and any other suitable method to find a mismatch and correct the mismatch may be implemented in accordance with an embodiment.

In an embodiment, mismatched prints are associated with various characteristics. For instance, Prints may be internally mismatched, as are compound prints such as the Pocket Loop, Arch Loop and Arch Whorl. Internally mismatched prints may be associated with signs of early learning delays as the child must learn to view the world not just through the ten lenses illustrated by the fingers but through the multiple lenses identified by the fingerprints. For instance, internal mismatches may be associated with signs of possible dyslexia. Additional information is described above, such s in connection with FIGS. 22-27.

As another example, mismatched prints including a whorl on the right thumb and a loop on the left thumb may be associated with characteristics related to people who are competitive at work or in school but has no desire to fight at home or vise versa if the prints are reversed. Advice may be associated with mismatched prints (or other hand characteristics) and provided to users. For instance, advice to avoid work out of home without complete separation of home office or advice to do homework in library or study hall may be given to people with the above mismatched thumb prints.

As yet another example, certain mismatched prints may be associated with challenges in movement. For instance, when prints do not match finger for finger on both hands, there may be subtle issues with coordination that may require special training and coaching to compete in sports. Besides indicating differences in behavioral characteristics in different aspects of the subject's life, one will also probably observe, through the most precise measurements, a slight (split second) difference in left and right hand action and reaction times that will cause coordination problems with two handed basketball shots going off the rim, or golf shots that tend to slice or hook, for example. Their effects may be correctable through training, such as training the basketball player to use a one handed push shot at the free throw line, but should always be measurable and will be subtly present. Because of the subtle imbalance this can cause in sports, further study should be made by trainers and others in sports medicine as well as coaches.

In another instance, “competitors” (who hate to loose) are distinguished from those who strive to accomplish a goal and are associated with whorls on fingers 2, 3, 4, and 5. The first competitors may be associated with characteristics related to those that seek and will fight to “win” against all who they believe they can defeat. The other competitors seek to excel in their given or chosen tasks, and thus they may appear to be competitors, but without the whorl on the thumb, such people may be identified as ones who really only mean to excel.

Referring to FIG. 102C, in one embodiment, Providing Personality Characteristics 10228 is the concluding step of the process 10200. Providing may include, for example, communicating the personality characteristic information to the client for display. Communicating for display may include, for example, providing a result set from, for example, a query request to, for example, the business logic portion of web page (i.e. a JavaBean) for processing and display in the web browser, in accordance with an embodiment. Various other methods may also be used, in accordance with at least various other methods to acquire hand characteristic information, within the scope of one or more embodiment of the disclosure.

The process of FIGS. 102A-C may contain additional or fewer steps or may accomplish each step in any suitable manner in accordance with an embodiment of the disclosure. In addition, in various embodiments, the order of at least some steps of FIGS. 102A-C may be performed in an order different than that which is explicitly disclosed and some steps may be performed simultaneously.

Data Store

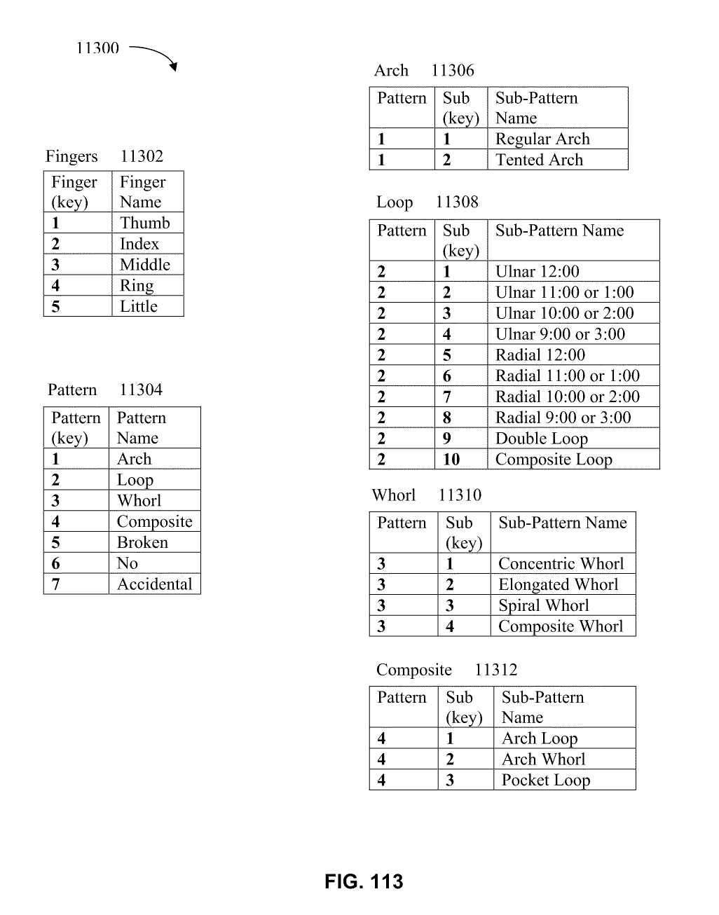

FIGS. 113-118 show an embodiment of a database schema that may be utilized in the data store 10110 of FIG. 101. The exemplary schema is only a sample and should not be considering limiting to the scope of the disclosure. In an embodiment, the sample environment of FIG. 101 may utilize the sample database schema of FIGS. 113-118 to implement the process of FIGS. 102A-C, or variations thereof. In an embodiment, the data store may be a relational database, such as IBM DB2, MySQL, Oracle 8i/9i, etc., an object-oriented database, or any other type of suitable data store. Various combinations of the examples and/or embodiments, and other technologies and/or methods that associate hand characteristics with personality characteristics may be utilized.

FIG. 113 shows an embodiment of example of a set 11300 of database tables that may be utilized to store hand characteristic information in association with personality characteristic information. It should be noted that FIG. 113 provides an example of tables for a relational database according to a particular schema, but that other types of databases and other schemas are contemplated as being within the scope of the present disclosure. In the illustrated embodiment, one or more tables may have one or more primary keys. A primary key is a column, or a set of columns, that uniquely identifies each record in a table. In an embodiment, one or more tables may also possess one or more foreign keys. A foreign key is a column, or a set of columns, that refers to a column, or set of columns, in another table.

Referring to FIG. 113, in an embodiment, Finger table 11302 is an example of a table storing finger information with the column “Finger” as a sample primary key. Pattern table 1304 is an example of a table storing pattern information with the column “Pattern” as a sample primary key. Arch table 1306 is an example of a table storing sub-pattern information of an arch pattern with the column “Sub” as a sample primary key and the column “Pattern,” referring to the “Pattern” column from table 1304, as a sample foreign key. Loop table 1308 is an example of a table storing sub-pattern information of a loop pattern with the column “Sub” as a sample primary key and a column “Pattern,” referring to the “Pattern” column from table 1304, as the sample foreign key. Whorl table 1310 is an example of a table storing sub-pattern information of a whorl pattern with a column “Sub” as the sample primary key and a column “Pattern,” referring to the “Pattern” column from table 1304, as the sample foreign key. Composite table 1312 is an example of a table storing sub-pattern information of composite patterns with the column “Sub” as a sample primary key and a column “Pattern,” referring to the “Pattern” column from table 1304, as the sample foreign key. As noted, the tables, columns, and records of the tables of FIG. 113 are mere examples of a database schema. Various combinations of tables, columns, and/or records, and other suitable schema, including various primary and/or foreign key designs, are within the scope of one or more embodiments of the disclosure.

As noted, the tables in FIG. 113 are, in an embodiment, relational database tables. User input may be used to locate among the tables information that is relevant to a particular hand characteristic or set of characteristics. For instance, if a user input information corresponding to a loop pattern, a computer system implementing an embodiment of the invention may use the primary key of the table 11302 to determine that the loop pattern, according to the table 11302, refers to the table 11308. As seen in the figures, other tables and entries in the tables refer to other tables.

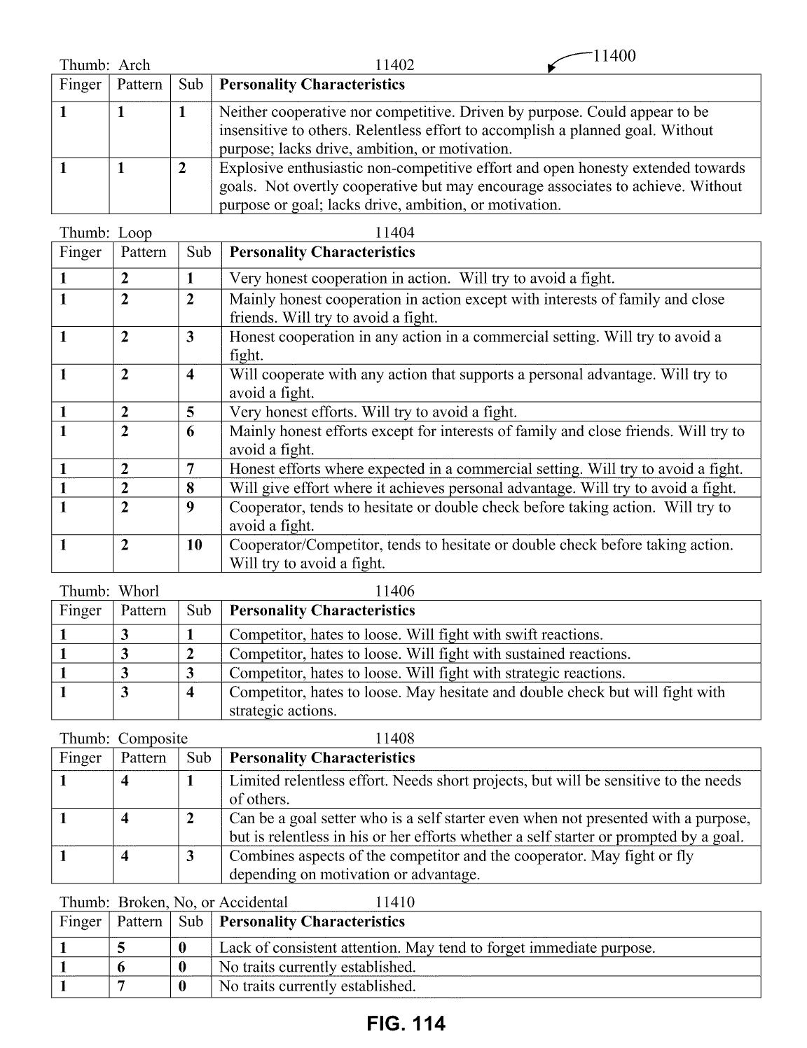

FIGS. 114-118 show an embodiment of exemplary tables of example database records associating sample dermatoglyphic patterns with sample personality characteristics. For instance, the tables 11400 of FIG. 114 show an embodiment of dermatoglyphic patterns found on the thumb and example associated personality characteristics.

In FIG. 114, in an embodiment, the example Thumb: Arch table 11402 shows example personality characteristics that may be associated with arch patterns of the thumb. The example Thumb: Loop table 11404 shows example personality characteristics that may be associated with loop patterns of the thumb. The example Thumb: Whorl table 11406 shows example personality characteristics that may be associated with whorl patterns of the thumb. The example Thumb: Composite table 11408 shows example personality characteristics that may be associated with composite patterns of the thumb. And the example Thumb: Broken, No or Accidental table 11410 shows example personality characteristics that may be associated with broken, no, or accidental patterns of the thumb.

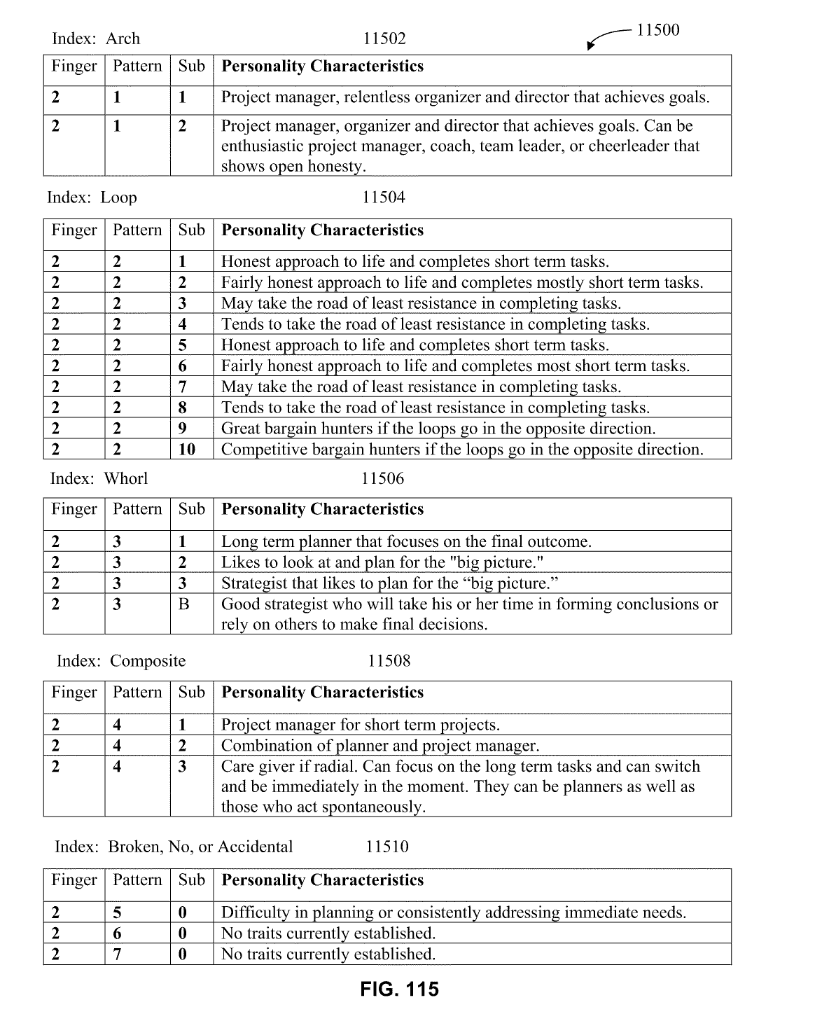

Each of the sample tables 11402, 11404, 11406, 11408, and 11410, in an embodiment, has a primary key and three foreign keys. The example primary key of the tables 11402, 11404, 11406, 11408, and 11410 is the column “Sub.” The example foreign keys are “Finger,” referring to the column “Finger” from table 1302 of FIG. 113, “Pattern,” referring to the column “Pattern” from table 1304 of FIG. 113, and “Sub” referring to the columns “Sub” from tables 11306, 11308, 11310, and 11312 of FIG. 113. In another embodiment, not shown in the Figures, tables 11402, 11404, 11406, 11408, and 11410 may be combined to form a single table. For this embodiment, the primary key for the combined table would be the set of columns “Sub” and “Pattern,” since each record would have a unique value for these two columns combined. A similar combination and composite primary key may be utilized for one or more the tables of FIGS. 115-118, in an embodiment. Various combinations of tables, columns, and/or records, and other suitable schema, including various primary and/or foreign key designs, are within the scope of one or more embodiments of the disclosure.

In an embodiment, the exemplary foreign key columns of tables 11402, 11404, 11406, 11408, and 11410 are utilized to manage associations. For instance, the first exemplary record of table 11402 contains the value “1” for the column “Finger.” As detailed above, the foreign key column “Finger” of table 11402 refers to the column “Finger” of table 11302 of FIG. 113. In table 11302 of FIG. 113, the record that is uniquely identified by the value “1” in the column “Finger” contains the string “Thumb” in the column “Finger Name.” This shows that, in an embodiment, the value “1,” when used as an entry in a foreign key column referring to the column “Finger” of table 11302, corresponds to the finger name “Thumb.”

Returning to FIG. 114, the first exemplary record of table 11402 also contains the value “1” for the column “Pattern.” As detailed above, the foreign key column “Pattern” of table 11402 refers to the column “Pattern” of table 11304 of FIG. 113. In table 11304 of FIG. 113, the record that is uniquely identified by the value 1 in the column “Pattern” contains the string “Arch” in the column “Pattern Name.” This shows that, in an embodiment, the value “1,” when used as an entry in a foreign key column referring to the column “Pattern” of table 11304, corresponds to the pattern name “Arch.”

Returning again to FIG. 114, the first exemplary record of table 11402 also contains the value “1” for the column “Sub.” As detailed above, the foreign key column “Sub” of table 11402 refers to the columns “Sub” of tables 11306, 11308, 11310, and 11312 of FIG. 113. In tables 1306, 11308, 11310, and 11312 of FIG. 113, the record that is uniquely identified by the value “1” in the column “Pattern” and the value “1” in column “Sub” contains the string “Regular Arch” in the column “Pattern Name.” This shows that the values “1” and “1,” in an embodiment, when used as entries in foreign key columns referring to the column “Pattern” and the column “Sub” of tables 11306, 11308, 11310, and 11312, correspond to the sub-pattern name “Regular Arch.”

Returning again to FIG. 114, the exemplary record from table 11402 that is identified by the values “1” for the column “Finger,” “1” for the column “Pattern,” and “1” for the column “Sub,” also contains the string “Neither cooperative nor competitive. Driven by purpose. Could appear to be insensitive to others. Relentless effort to accomplish a planned goal. Without purpose lacks drive, ambition, or motivation” for the column “Personality Characteristics.” This shows that the sub-pattern “Regular Arch” of the dermatoglyphic pattern “Arch” on the finger “Thumb” may be associated with the personality characteristics “Neither cooperative nor competitive. Driven by purpose. Could appear to be insensitive to others. Relentless effort to accomplish a planned goal. Without purpose, lacks drive, ambition, or motivation,” in an embodiment. The remaining entries for each of the FIGS. 114-118, and each of the tables for each of the figures, contain similar pattern to personality characteristics exemplary associations. These exemplary associations, and various combinations, are within the scope of one or more embodiments of the disclosure.

The tables 11500 of FIG. 115, in an embodiment, show example dermatoglyphic patterns found on the index finger and example associated personality characteristics. For instance, in an embodiment, the example Index: Arch table 11502 shows example personality characteristics that may be associated with arch patterns of the index finger. The example Index: Loop table 11504 shows example personality characteristics that may be associated with loop patterns of the index finger. The example Index: Whorl table 11506 shows example personality characteristics that may be associated with whorl patterns of the index finger. The example Index: Composite table 11508 shows example personality characteristics that may be associated with composite patterns of the index finger. And the example Index: Broken, No or Accidental table 11510 shows example personality characteristics that may be associated with broken, no, or accidental patterns of the index finger.

In an embodiment, the exemplary foreign key columns of tables 11502, 11504, 11506, 11508, and 11510 are utilized to manage associations in a similar manner as discussed above for FIG. 114. For instance, the exemplary record from table 11502 that is identified by the values “2” for the column “Finger,” “1” for the column “Pattern,” and “1” for the column “Sub,” contains the string “Project manager, relentless organizer and director that achieves goals” for the column “Personality Characteristics.” This shows that the sub pattern “Regular Arch” of the dermatoglyphic pattern “Arch” on the finger “Index” may be associated with the personality characteristics “Project manager, relentless organizer and director that achieves goals,” in an embodiment. The remaining entries for each of the FIGS. 114-118, and each of the tables for each of the figures, contain similar pattern to personality characteristics exemplary associations. These exemplary associations, and various combinations, are within the scope of one or more embodiments of the disclosure.

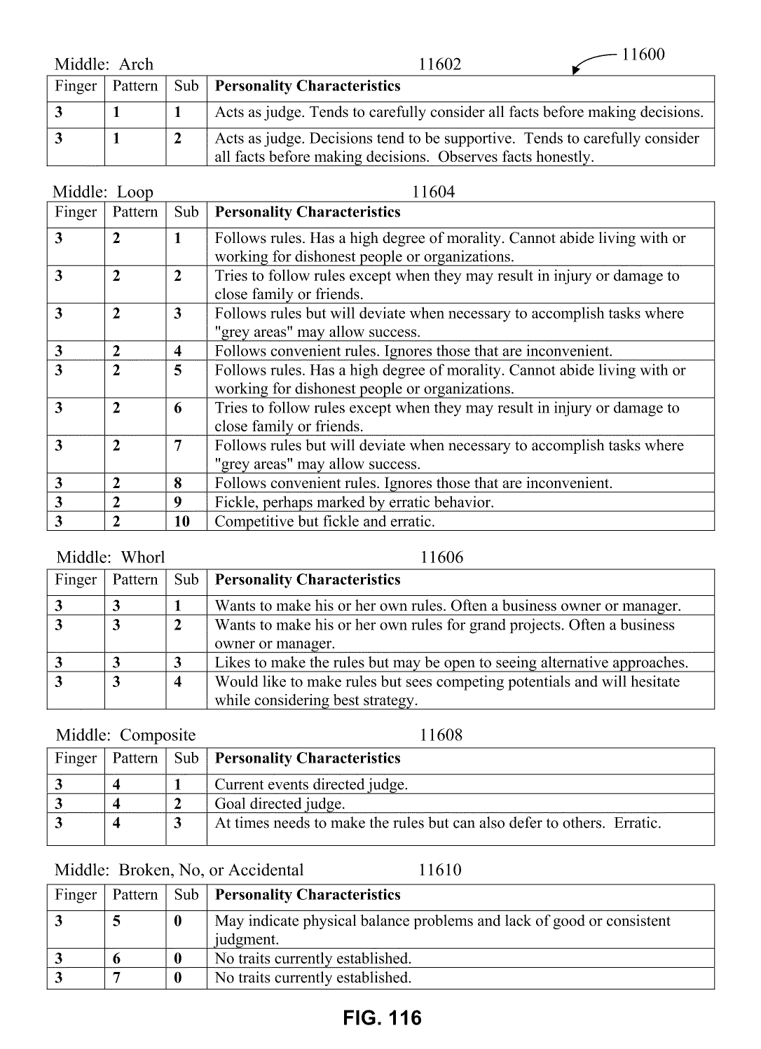

The tables 11600 of FIG. 116, in an embodiment, show example dermatoglyphic patterns found on the middle finger and example associated personality characteristics. For instance, in an embodiment, the example Middle: Arch table 11602 shows example personality characteristics that may be associated with arch patterns of the middle finger. The example Middle: Loop table 11604 shows example personality characteristics that may be associated with loop patterns of the middle finger. The example Middle: Whorl table 11606 shows example personality characteristics that may be associated with whorl patterns of the middle finger. The example Middle: Composite table 11608 shows example personality characteristics that may be associated with composite patterns of the middle finger. And the example Middle: Broken, No or Accidental table 11610 shows example personality characteristics that may be associated with broken, no, or accidental patterns of the middle finger.

In an embodiment, the exemplary foreign key columns of tables 11602, 11604, 11606, 11608, and 11610 are utilized to manage associations in a similar manner as discussed above for FIG. 114. For instance, the exemplary record from table 11602 that is identified by the values “3” for the column “Finger,” “1” for the column “Pattern,” and “1” for the column “Sub,” contains the string “Acts as a judge. Tends to carefully consider all facts before making decisions” for the column “Personality Characteristics.” This shows that the sub pattern “Regular Arch” of the dermatoglyphic pattern “Arch” on the finger “Middle” may be associated with the personality characteristics “Acts as a judge. Tends to carefully consider all facts before making decisions,” in an embodiment. The remaining entries for each of the FIGS. 114-118, and each of the tables for each of the figures, contain similar pattern to personality characteristics exemplary associations. These exemplary associations, and various combinations, are within the scope of one or more embodiments of the disclosure.

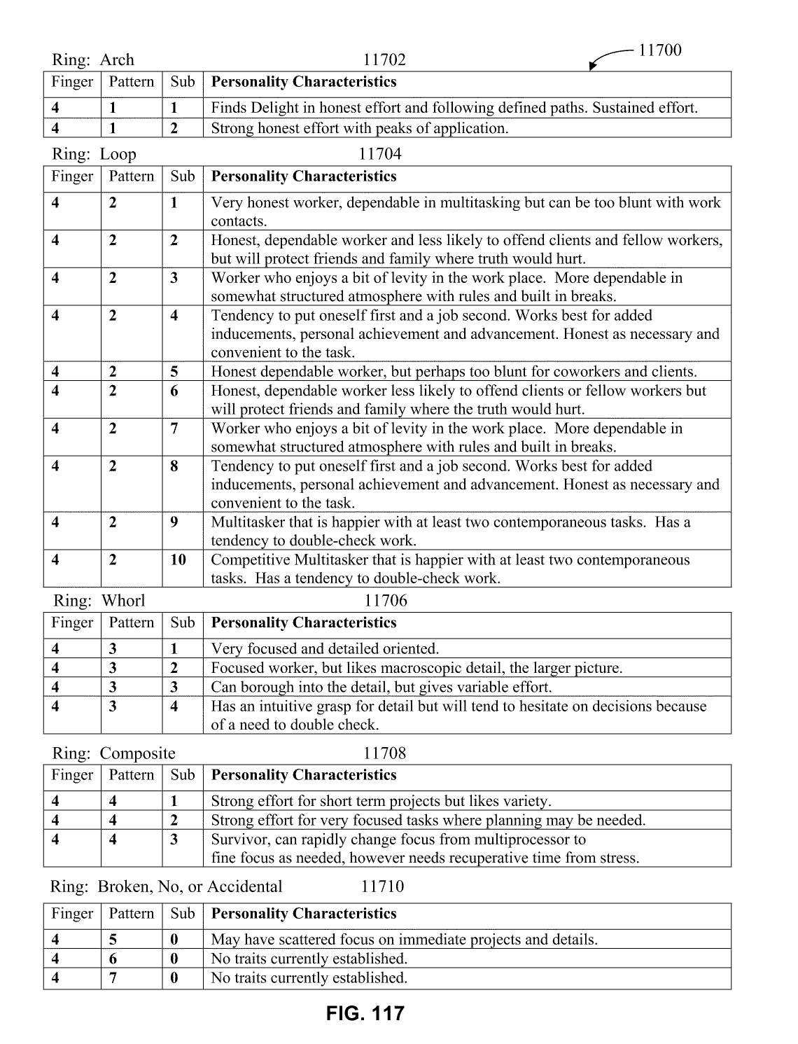

The tables 11700 of FIG. 117, in an embodiment, show example dermatoglyphic patterns found on the ring finger and example associated personality characteristics. For instance, in an embodiment, the example Ring: Arch table 11702 shows example personality characteristics that may be associated with arch patterns of the ring finger. The example Ring: Loop table 11704 shows example personality characteristics that may be associated with loop patterns of the ring finger. The example Ring: Whorl table 11706 shows example personality characteristics that may be associated with whorl patterns of the middle finger. The example Ring: Composite table 11708 shows example personality characteristics that may be associated with composite patterns of the ring finger. And the example Ring: Broken, No or Accidental table 11710 shows example personality characteristics that may be associated with broken, no, or accidental patterns of the middle finger.

In an embodiment, the exemplary foreign key columns of tables 11702, 11704, 11706, 11708, and 11710 are utilized to manage associations in a similar manner as discussed above for FIG. 114. For instance, the exemplary record from table 11702 that is identified by the values “4” for the column “Finger,” “1” for the column “Pattern,” and “1” for the column “Sub,” contains the string “Finds delight in honest effort and following defined paths. Gives sustained effort” for the column “Personality Characteristics.” This shows that the sub-pattern “Regular Arch” of the dermatoglyphic pattern “Arch” on the finger “Ring” may be associated with the personality characteristics “Finds delight in honest effort and following defined paths. Gives sustained effort,” in an embodiment. The remaining entries for each of the FIGS. 114-118, and each of the tables for each of the figures, contain similar pattern to personality characteristics exemplary associations. These exemplary associations, and various combinations, are within the scope of one or more embodiments of the disclosure.

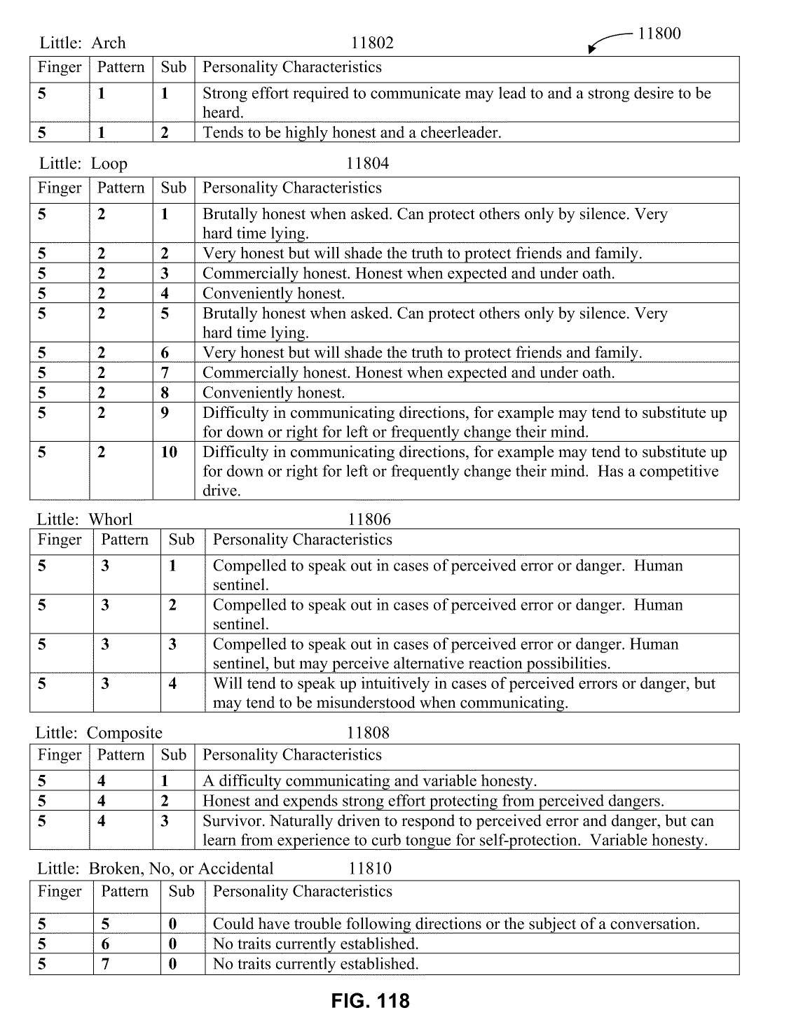

The tables 11800 of FIG. 118, in an embodiment, show example dermatoglyphic patterns found on the little finger and example associated personality characteristics. For instance, in an embodiment, the example Little: Arch table 11802 shows example personality characteristics that may be associated with arch patterns of the little finger. The example Little: Loop table 11804 shows example personality characteristics that may be associated with loop patterns of the little finger. The example Little: Whorl table 11806 shows example personality characteristics that may be associated with whorl patterns of the little finger. The example Little: Composite table 11808 shows example personality characteristics that may be associated with composite patterns of the little finger. And the example Little: Broken, No or Accidental table 11810 shows example personality characteristics that may be associated with broken, no, or accidental patterns of the little finger.

In an embodiment, the exemplary foreign key columns of tables 11802, 11804, 11806, 11808, and 11810 are utilized to manage associations in a similar manner as discussed above for FIG. 114. For instance, the exemplary record from table 11802 that is identified by the values “5” for the column “Finger,” “1” for the column “Pattern,” and “1” for the column “Sub,” contains the string “Requires a strong effort to communicate which may lead to reticence and/or verbosity and a strong desire to be heard” for the column “Personality Characteristics.” This shows that the sub-pattern “Regular Arch” of the dermatoglyphic pattern “Arch” on the finger “Little” may be associated with the personality characteristics “Requires a strong effort to communicate which may lead to reticence and/or verbosity and a strong desire to be heard,” in an embodiment. The remaining entries for each of the FIGS. 114-118, and each of the tables for each of the figures, contain similar pattern to personality characteristics exemplary associations. These exemplary associations, and various combinations, are within the scope of one or more embodiments of the disclosure.

Data Store Process

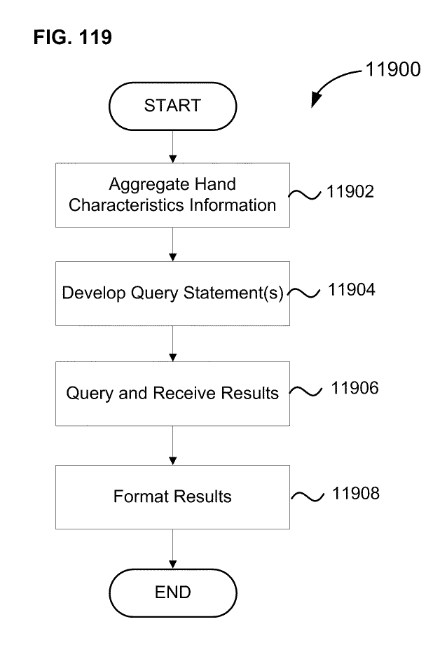

In an embodiment, FIG. 119 shows a sample interaction process 11900 that may be performed between the Data Store 10110 of FIG. 101 and the Application server 10108 of FIG. 101. The interaction process, 11900, may take place, for example, during step 10224, Retrieving Information, of FIG. 102C, in an embodiment.

In an embodiment, FIG. 119 may begin with step 11902, Aggregating Hand Characteristic Information. Aggregating may include, in this example, at the Application Server 10108 of FIG. 101, compiling at least part of the hand characteristic information that has been acquired through previous steps 10202, 10204, 10206, 10208, 10210, 10212, 10214, 10216, and 10218 of FIGS. 102A-B. Compiling may include organizing hand characteristic information by the finger, by the hand characteristic, by the hand, or by any other suitable organization, in an embodiment. A combination of various organizations is also within the scope of the disclosure. The aggregating may be done at various other elements of FIG. 101 or any other suitable location, in accordance with an embodiment. Various other methods may also be used, in accordance with at least various other methods to acquire hand characteristic information, within the scope of one or more embodiment of the disclosure.

In an embodiment, implementation of the process 11900 may include proceeding next to step 11904, Developing Query Statements. Developing may include, in this example, at the Application Server 10108 of FIG. 101, utilizing the aggregated hand characteristic from step 11902 and forming one or more query statements in accordance with a sample database schema, in an embodiment. Developing may further include forming one or more query statements organized by the finger, by the hand characteristic, by the hand, or by any other suitable organization, in an embodiment. A combination of various organizations is also within the scope of the disclosure. The one or more query statements formed may utilize, for example, SQL, XML, SCSI, iSCSI, and any other suitable query/machine languages. The developing may be done at various other elements of FIG. 101 or any other suitable location, in accordance with an embodiment. Various other methods may be used, in accordance with at least various other methods to acquire hand characteristic information and/or aggregate hand characteristic information, within the scope of one or more embodiment of the disclosure.

In an embodiment, implementation of the process 11900 may include proceeding next to step 11906, Querying and Receiving Results. Querying may include, in this example, at the data store 10110 of FIG. 101, utilizing the one or more query statements formed from step 11902 and querying one or more data stores in accordance with a sample database schema, in an embodiment. Receiving may include, for example, at the Application Server 10108 of FIG. 101, receiving a result set of personality characteristic information from the data store 10110 of FIG. 1 based on at least the one or more query statements, in an embodiment. The querying and/or receiving may be done at various other elements of FIG. 101 or any other suitable location, in accordance with an embodiment. Various other methods may be used, in accordance with at least various other methods to acquire hand characteristic information and/or aggregate hand characteristic information and/or develop query statements, within the scope of one or more embodiment of the disclosure.

In an embodiment, implementation of the process 11900 may include proceeding next to step 11908, Formatting Results. Formatting may include, in this example, at the Application Server 10108, formatting the personality characteristics information to a format conducive to the step 10226 of FIG. 102C, Correcting Conflicts. Formatting may further include organizing personality characteristic information according to one or more of tendencies in action, tendencies in communication, tendencies at work, tendencies at home, tendencies in deliberation, tendencies in judgment, tendencies in honesty, tendencies in intelligence, associations with hand characteristics, or any other suitable organization. A combination of various organizations is also within the scope of the disclosure. The formatting may be done at various other elements of FIG. 101 or any other suitable location, in accordance with an embodiment. Various other methods may be used, in accordance with at least various other methods to acquire hand characteristic information and/or aggregate hand characteristic information and/or develop query statements and/or query a database and receive results, within the scope of one or more embodiment of the disclosure.

In an embodiment, implementation of the process 11900 may include proceeding next to step 10226 of FIG. 102C, Correcting conflicts. Correcting conflicts has been described above. The process of FIG. 119 may contain additional or fewer steps or may accomplish each step in any suitable manner in accordance with an embodiment of the disclosure. Once the results are formatted, in an embodiment, the results are presented to a user, in accordance with an embodiment. Presenting the results to a user may include, for instance, providing information corresponding to the results for display on a user device. As an example, a hypertext markup language (HTML) file having the formatted results may be sent to a user device over a network. Generally, any way of providing results to the user may be utilized.

Finger Codes

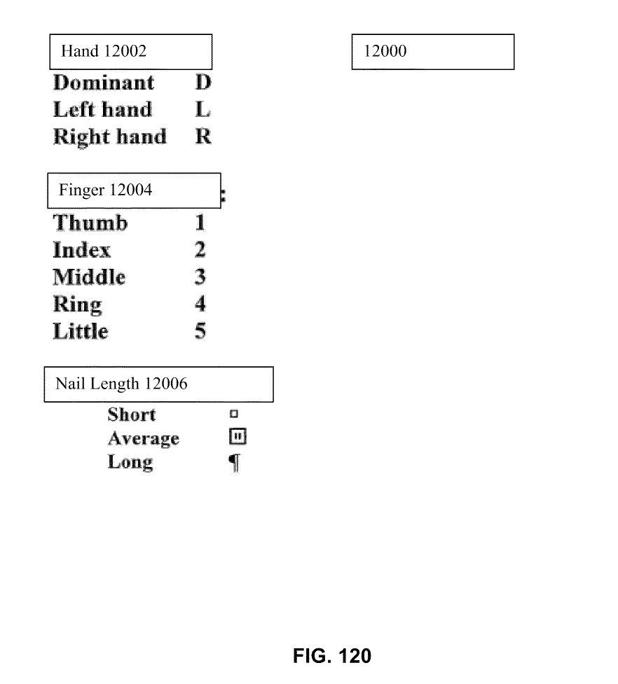

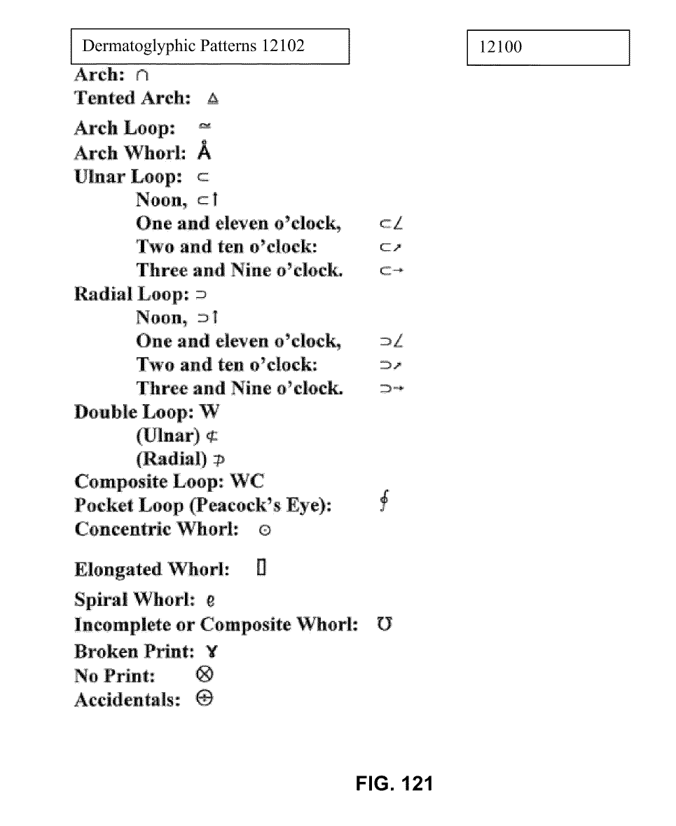

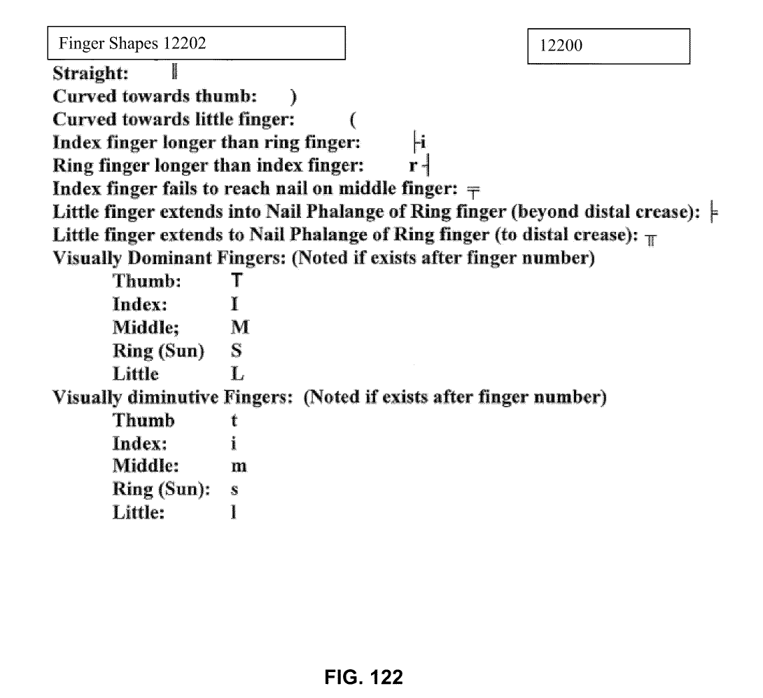

FIGS. 120-122 show examples of short-hand code 12000, 12100, and 12200 for hand characteristics in accordance with an embodiment of the invention. The exemplary short-hand code can be used to set up automated programs for fast hand characteristics acquisition. For instance, example codes 12002 of FIG. 120 can identify hand dominance (left or right) and hand type (left or right) in accordance with an embodiment. Example codes 12004 of FIG. 120 can identify a finger (thumb, index, middle, ring, or little) in accordance with an embodiment. Example codes 12006 of FIG. 120 can identify a nail length (short, average, or long), in accordance with an embodiment. Examples codes 12102 can identify a dermatoglyphic pattern (arch, tented arch, arch loop, etc.) in accordance with an embodiment. And example codes 12202 can identify finger shapes (straight, curved toward thumb, curved toward little finger, etc.) in accordance with an embodiment. Further embodiments may include other suitable hand characteristics and/or other suitable codes for disclosed hand characteristics or other suitable hand characteristics, or any combinations.

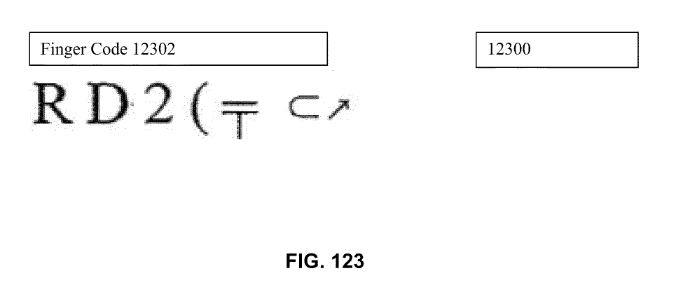

FIG. 123 shows exemplary short hand code 12300 in accordance with an embodiment. For example, Finger Code 12302, in an embodiment, shows code that may represent an index finger on the right, dominant, hand that is shorter than the ringer finger, that does not reach the distal crease of the middle finger, that is curved towards the little finger, and that has a 2:00 ulnar loop dermatoglyphic pattern. Further embodiments of codes may include an aggregate of code for one or more of the hand characteristics for one or more of the fingers on one or both hands, or any combinations.

Data Gathering

Various techniques may be used to acquire hand characteristic information that may be utilized for various embodiments of the disclosure. Techniques described herein may be used, for instance, to identify dermatoglyphic patterns, finger flexibility, finger morphology, knuckle dimensions, finger dimensions, and any others suitable observations. Sample hand characteristics acquisition methods in accordance with various embodiments are further described in the following paragraphs.

Dermatoglyphic pattern information may be acquired by a variety of methods. For instance, in an embodiment, visual recording may be utilized in good light by the naked eye and/or by a magnifying glass with, for example, a 5× magnification. Mechanical methods, such as clay impressions, sticky paper placed over rollers, or ink print recording, may also be utilized in an embodiment. Other mechanical methods that are within the scope of one or more embodiments may include the methods found in the U.S. Department of Justice, Federal Bureau of Investigation Manual: The Science of Fingerprints (rev. 12-84) Chapter 1× and following chapters. Optical methods, such as film using high resolution cameras and lenses that allow the conversion of patterns to electronic signals through the use of digital cameras or scanners, may also be utilized in an embodiment. Frustrated Total Internal Reflection (FTIR) devices may be an example of a scanner device. There are other methods of introducing light into the fingerprint, usually from the side, that allow for sufficient contrast to capture the ridge and valley details of the prints within an embodiment of the disclosure. Electrical methods, such as conductivity measurements, capacitance measurements, and RF (radio frequency) measurements, may also be utilized in an embodiment. An advantage of RF-based sensors is that they can be confined to generate images of the internal, still growing live layers of the skin. Ultrasound methods, where ultrasonic energy is used to detect fingerprint patterns based upon the different absorption rates of ridges and valleys or upon echo reflection techniques, may also be utilized in an embodiment. Thermal methods, where the thermal energy flow between a touched sensor and finger ridges can distinguish ridges from valleys, may also be utilized in an embodiment. Various others techniques may be utilized to acquire dermatoglyphic pattern information within the scope of one or more embodiments of the disclosure.

Finger flexibility and finger morphology information may be acquired by a variety of methods. In an embodiment, flexibility may be determined by gentle pressure on the fingers, individually, principally establishing the normal limits for dorsal abduction of the index, middle, ring, and little fingers and the lateral abduction between the index finger and thumb. This may be observed, for example, by unaided visual recognizance or by use of a protractor, as shown in FIGS. 33-35, and 67. In an embodiment, finger flexibility may also be acquired by placing the palm down on a flat surface and measuring the angle of dorsal abduction beyond the center plain of the palm for each of the fingers. In an embodiment, the pressure used should be gentle, avoiding all pain and possible injury, and should stop when any significant resistance is reached. Acquisition maybe accomplished, in an example embodiment, physically, or through mechanical devices designed to measure pressure and resistance, such as a pressure glove. Various others techniques may be utilized to acquire finger flexibility or morphology information within the scope of one or more embodiments of the disclosure.

Finger and knuckle dimension information may be acquired by a variety of methods and in a variety of ways. For instance, in an embodiment, each finger may be measured along the palmar side from the proximal phalange crease to the flesh tip of the nail phalange on the same finger. Phalange length may be measured, for example, along the palmar side from the most proximal phalange crease to the next crease, or in the case of the distal phalange, to fleshy tip of the distal phalange, in an embodiment. Phalange width may be measured, for example, horizontally across the midpoint of the length of the phalange with the fingers held vertically, in an embodiment. Knuckle width may be measured, for example, horizontally across the midpoint of the length of the knuckle with the fingers held vertically on a flat surface, in an embodiment. The length of the hand may be measured, for example, with the hand flat from the midpoint of the palmar wrist crease to the midpoint of the most proximal phalange crease on the middle finger, in an embodiment. The width of the hand may be measured, for example, with the hand held flat horizontally from a point where the thumb joins the palm along the radial edge below the index finger to the opposite point on the ulnar edge of the hand below the little finger, in an embodiment. Various others techniques may be utilized to acquire finger and knuckle dimension information within the scope of one or more embodiments of the disclosure.

In an embodiment, a computer configured with code may be utilized to model a subject's hand using 3-dimensional modeling techniques. The modeling may be used to determine one or more of, dermatoglyphic patterns, finger flexibility, finger morphology, knuckle dimensions, relative finger length, finger dimensions, and any others suitable hand observations, in accordance with an embodiment. The modeling may also be used to model, for visual display, one or more of dermatoglyphic patterns, finger flexibility, finger morphology, knuckle dimensions, relative finger length, finger dimensions, and any others suitable hand observations, in accordance with an embodiment. The modeling may be provided for display to the user along with the set of personality characteristics, in an embodiment. The modeling along side the set of personality characteristics may, in an embodiment, show the user the associations between the modeled hand characteristics and the associated personality characteristics. The modeling code may include any suitable code capable of modeling the hand characteristics utilized in the embodiments of the disclosure.

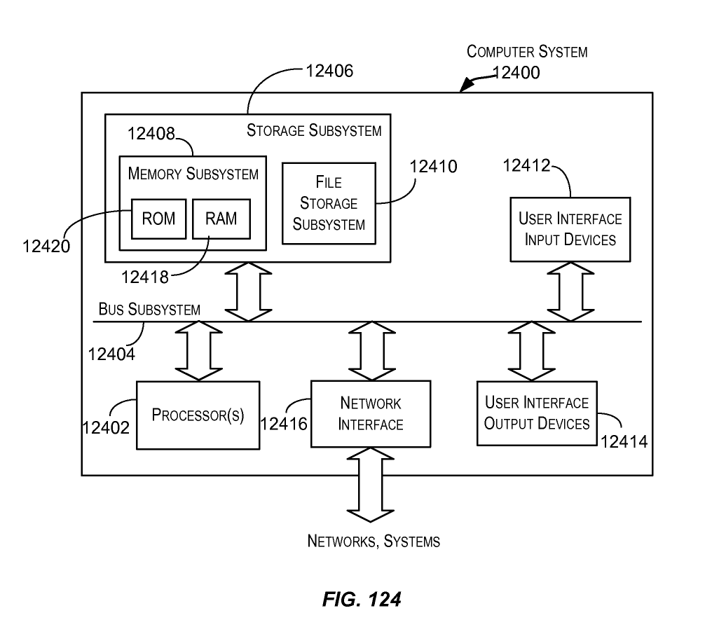

FIG. 124 is a simplified block diagram of a computer system 12400 that may be used to practice an embodiment of the present invention. In various embodiments, computer system 12400 may be used to implement any of the systems illustrated and described above. For example, computer system 12400 may be used to implement processes for style checking according to the present disclosure. As shown in FIG. 124, computer system 12400 includes a processor 12402 that communicates with a number of peripheral subsystems via a bus subsystem 12404. These peripheral subsystems may include a storage subsystem 12406, comprising a memory subsystem 12408 and a file storage subsystem 12410, user interface input devices 12412, user interface output devices 12414, and a network interface subsystem 12416.

Bus subsystem 12404 provides a mechanism for enabling the various components and subsystems of computer system 12400 to communicate with each other as intended. Although bus subsystem 12404 is shown schematically as a single bus, alternative embodiments of the bus subsystem may utilize multiple busses.

Network interface subsystem 12416 provides an interface to other computer systems and networks. Network interface subsystem 12416 serves as an interface for receiving data from and transmitting data to other systems from computer system 12400. For example, network interface subsystem 12416 may enable a user computer to connect to the Internet and facilitate communications using the Internet in order to provide information corresponding to hand characteristics and, in response, receive a personality profile.

User interface input devices 12412 may include a keyboard, pointing devices such as a mouse, trackball, touchpad, or graphics tablet, a scanner, a barcode scanner, a touch screen incorporated into the display, audio input devices such as voice recognition systems, microphones, and other types of input devices. In general, use of the term “input device” is intended to include all possible types of devices and mechanisms for inputting information to computer system 12400. An input device may be used, for example, to allow users to input information corresponding to hand characteristics.

User interface output devices 12414 may include a display subsystem, a printer, a fax machine, or non-visual displays such as audio output devices, etc. The display subsystem may be a cathode ray tube (CRT), a flat-panel device such as a liquid crystal display (LCD), or a projection device. In general, use of the term “output device” is intended to include all possible types of devices and mechanisms for outputting information from computer system 12400. A personality profile may be output using one or more of user interface output devices 12414.

Storage subsystem 12406 provides a computer-readable storage medium for storing the basic programming and data constructs that provide the functionality of the present invention. Software (programs, code modules, instructions) that when executed by a processor provide the functionality of the present invention may be stored in storage subsystem 12406. These software modules or instructions may be executed by processor(s) 12402. Storage subsystem 12406 may also provide a repository for storing data used in accordance with the present invention. Storage subsystem 12406 may comprise memory subsystem 12408 and file/disk storage subsystem 12410.

Memory subsystem 12408 may include a number of memories including a main random access memory (RAM) 12418 for storage of instructions and data during program execution and a read only memory (ROM) 12420 in which fixed instructions are stored. File storage subsystem 12410 provides a non-transitory persistent (non-volatile) storage for program and data files, and may include a hard disk drive, a floppy disk drive along with associated removable media, a Compact Disk Read Only Memory (CD-ROM) drive, an optical drive, removable media cartridges, and other like storage media. Information corresponding to hand characteristics and their associations with various behavioral characteristics may be stored by the memory subsystem 12408, as well as executable instructions for performing any processes described herein, or variations thereof.

Computer system 12400 can be of various types including a personal computer, a portable computer, a workstation, a network computer, a mainframe, a kiosk, a server or any other data processing system. Due to the ever-changing nature of computers and networks, the description of computer system 12400 depicted in FIG. 124 is intended only as a specific example for purposes of illustrating the preferred embodiment of the computer system. Many other configurations having more or fewer components than the system depicted in FIG. 124 are possible.

Although specific embodiments of the invention have been described, various modifications, alterations, alternative constructions, and equivalents are also encompassed within the scope of the invention. Embodiments of the present invention are not restricted to operation within certain specific data processing environments, but are free to operate within a plurality of data processing environments. Additionally, although embodiments of the present invention have been described using a particular series of transactions and steps, it should be apparent to those skilled in the art that the scope of the present invention is not limited to the described series of transactions and steps.

Although specific embodiments have been described, various modifications, alterations, alternative constructions, and equivalents are also encompassed within the scope of the invention. Embodiments of the present invention are not restricted to operation within certain specific data processing environments, but are free to operate within a plurality of data processing environments. Additionally, although embodiments of the present invention have been described using a particular series of transactions and steps, it should be apparent to those skilled in the art that the scope of the present invention is not limited to the described series of transactions and steps.

Further, while embodiments of the present invention have been described using a particular combination of hardware and software, it should be recognized that other combinations of hardware and software are also within the scope of the present invention. Embodiments of the present invention may be implemented only in hardware, or only in software, or using combinations thereof.

The specification and drawings are, accordingly, to be regarded in an illustrative rather than a restrictive sense. It will, however, be evident that additions, subtractions, deletions, and other modifications and changes may be made thereunto without departing from the broader spirit and scope of the invention.

Other variations are within the spirit of the present invention. Thus, while the invention is susceptible to various modifications and alternative constructions, certain illustrated embodiments thereof are shown in the drawings and have been described above in detail. It should be understood, however, that there is no intention to limit the invention to the specific form or forms disclosed, but on the contrary, the intention is to cover all modifications, alternative constructions, and equivalents falling within the spirit and scope of the invention, as defined in the appended claims.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected” is to be construed as partly or wholly contained within, attached to, or joined together, even if there is something intervening. The term “exemplary” is to be construed to indicate serving as an example, instance, or illustration and, unless otherwise indicated herein or clearly contradicted by context, does not imply preferred embodiments or desirability. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate embodiments of the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

All references, including publications, patent applications, and patents, cited herein are hereby incorporated by reference to the same extent as if each reference were individually and specifically indicated to be incorporated by reference and were set forth in its entirety herein.

What is claimed is:

1. A computer-implemented method for providing to a user an inventory set of personality characteristics derived from hand characteristics, the method comprising:
under the control of one or more computer systems configured with executable instructions,
storing, in a data store, a plurality of personality characteristics in a manner that associates the personality characteristics with corresponding hand characteristics, the hand characteristics including at least one of morphological characteristics and measurement characteristics;
receiving, based at least in part on a subject's hand, information that identifies a set of hand characteristics;
identifying, based at least in part on the received information, a set of personality characteristics from the data store; and
providing for display to the user information that is based at least in part on the identified set of personality characteristics.

2. The computer-implemented method of claim 1, wherein the hand characteristics further comprise one or more of the following:
identification of left or right;
hand dominance;
dermatoglyphic patterns for one or more fingers;
finger flexibility for one or more fingers;
finger placement at rest for one or more fingers;
finger morphology for one or more fingers;
finger absolute dimensions for one or more fingers;
finger relative dimensions for one or more fingers; and
nail dimensions for one or more fingers.

3. The computer-implemented method of claim 2, wherein the hand characteristics include dermatoglyphic patterns received from one or more of the following: visual recording, mechanical recording, optical recording, electrical recording, ultrasound recording, and thermal recording.

4. The computer-implemented method of claim 2, wherein the hand characteristics include finger flexibility characteristics that are based at least in part from data provided by a pressure glove.

5. The computer-implemented method of claim 1, wherein identifying a set of personality characteristics from the data store further comprises:
identifying mismatched personality characteristics identified based at least in part on the hand characteristics;
extrapolating new personality characteristics based at least in part on the hand characteristics; and
updating the plurality of personality characteristics in the data store.

6. The computer-implemented method of claim 5, wherein extrapolating the new personality characteristics is further based at least in part on a context of the hand characteristics.

7. The computer-implemented method of claim 1, wherein receiving the hand characteristics includes receiving the hand characteristics from a user interface comprising one or more forms.

8. The computer-implemented method of claim 1, wherein receiving the hand characteristics includes receiving the hand characteristics are received from at least one machine designed to determine hand characteristics.

9. The computer-implemented method of claim 1, further comprising modeling the hand characteristics are modeled using at least one three-dimensional modeling algorithm.

10. The computer-implemented method of claim 1, wherein the set of personality characteristics further comprises an inventory of personality characteristics.

11. A computer-readable storage medium, having stored thereon instructions that, when executed collectively by one or more processors, cause the one or more processors to:
store, in a data store, a plurality of personality characteristics in a manner that associates the personality characteristics with corresponding hand characteristics, the hand characteristics including at least one of morphological characteristics and measurement characteristics;
receive, based at least in part on a subject's hand, information that identifies a set of hand characteristics;
identify, based at least in part on the received information, a set of personality characteristics from the data store; and
provide for display to the user information that is based at least in part on the identified set of personality characteristics.

12. The computer-readable storage medium of claim 11, wherein the hand characteristics further comprise one or more of the following:
identification of left or right;
hand dominance;
dermatoglyphic patterns for one or more fingers;
finger flexibility for one or more fingers;
finger placement at rest for one or more fingers;
finger morphology for one or more fingers;
finger absolute dimensions for one or more fingers;
finger relative dimensions for one or more fingers; and
nail dimensions for one or more fingers.

13. The computer-readable storage medium of claim 11, wherein the instructions further include instructions that cause the one or more processors to
identify mismatched personality characteristics identified based at least in part on the hand characteristics;
extrapolate new personality characteristics based at least in part on the hand characteristics; and
update the plurality of personality characteristics in the data store.

14. The computer-readable storage medium of claim 11, wherein the instructions further include instructions that cause the one or more processors to provide a series of interface elements for receiving the hand characteristics, at least one of the interface elements being conditional on input received by one or more of the interface elements.

15. The computer-readable storage medium of claim 11, wherein the hand characteristics include one or more measurements based on a physical test of a hand.

16. A computer system for providing a personality inventory, comprising:
a data store that stores a plurality of personality characteristics in a manner that associates the personality characteristics with corresponding hand characteristics;
one or more processors; and
memory, including instructions that, when executed by the one or more processors, cause the one or more processors to:
receive, based at least in part on a subject's hand, information that identifies a set of hand characteristics, the hand characteristics including at least one of morphological characteristics and measurement characteristics;
identify, based at least in part on the received information, a set of personality characteristics from the data store; and
provide for display to the user information that is based at least in part on the identified set of personality characteristics.

17. The computer system of claim 16, wherein the hand characteristics further comprise one or more of the following:
identification of left or right;
hand dominance;
dermatoglyphic patterns for one or more fingers;
finger flexibility for one or more fingers;
finger placement at rest for one or more fingers;
finger morphology for one or more fingers;
finger absolute dimensions for one or more fingers;
finger relative dimensions for one or more fingers; and
nail dimensions for one or more fingers.

18. The computer system of claim 16, wherein the hand characteristics further comprise a plurality of the following:
identification of left or right;
hand dominance;
dermatoglyphic patterns for one or more fingers;
finger flexibility for one or more fingers;
finger placement at rest for one or more fingers;
finger morphology for one or more fingers;
finger absolute dimensions for one or more fingers;
finger relative dimensions for one or more fingers; and
nail dimensions for one or more fingers.

19. The computer system of claim 16, wherein the instructions further include instructions that cause the one or more processors to provide a plurality of interface elements for receiving the hand characteristics, at least one of the interface elements being conditional on input received by one or more of the interface elements.

20. The computer system of claim 16, wherein the data store associates one or more of the hand characteristics with honesty.
